(19) United States
(12) Patent Application Publication
(10) Pub. No.: US 2026/0207754 A1
(43) Pub. Date: Jul. 23, 2026

(54) MET PROTEIN DEGRADATION INDUCING COMPOUND

(71) Applicant: ONCOZEN CO., LTD., Cheongju-si, Chungcheongbuk-do (KR)

(72) Inventors: Joo-Hoon AHN, Seongnam-si (KR); Ho YOO, Seoul (KR); Sun-Hye LEE, Pohang-si (KR); Da-Hyun SONG, Incheon (KR)

(73) Assignee: ONCOZEN CO., LTD., Cheongju-si, Chungcheongbuk-do (KR)

(21) Appl. No.: 19/140,090

(22) PCT Filed: Dec. 21, 2023
(86) PCT No.: PCT/KR2023/021206

§ 371 Date: Jun. 17, 2025

(30) Foreign Application Priority Data
Dec. 27, 2022 (KR) 10-2022-0185429

(51) Int. Cl. A61K 47/55; A61K 47/54; A61P 35/00

(52) U.S. Cl. CPC A61K 47/55; A61K 47/545; A61P 35/00

(57) ABSTRACT

Compounds degrade target proteins based on chaperone-mediated protein degradation (CMPD) and methods of producing the same, and uses thereof. The present invention provides a target protein degradation compound that includes a first moiety (Chaperone Binding, CB) capable of binding to the chaperone protein or protein components of the chaperone complex, and a second moiety (Target Binding, TB) capable of binding to the target protein or proteins. Such a compound is structured in form of a chaperone binding part (CB)-linker (L)-target binding part (TB) or is a stereoisomer thereof. The compound of the present invention has the effect of inducing degradation of the MET protein based on CMPD technology. Therefore, the compound can be expected to exhibit superior anticancer effects by completely degrading the target protein (MET), thereby overcoming resistance and mutations associated with targeted anticancer drugs.

### TECHNICAL FIELD

The present invention relates to compounds that degrade target proteins based on chaperone-mediated protein degradation (CMPD) technology, methods of producing the same, and uses thereof.

### BACKGROUND ART

Cancer remains one of the unconquered diseases to this day despite the development of the first treatment in the 1940s. As cancer treatment technologies advance, the characteristics of anticancer drugs are continuously evolving. Approximately 40% of new drug research and development (R&D) investment is focused on anticancer drugs, resulting in the introduction of numerous new treatments. Despite these advancements, there is still an increasing unmet demand among cancer patients. The current direction of anticancer drug development is towards new anticancer drugs that overcome resistance or increase treatment response rates. Existing anticancer drugs depend on low molecular compounds, antibodies, and other existing technologies, with only 400 of the roughly 3,000 disease-causing genes targeted by approved drugs (85% undruggable targets). Furthermore, the introduction of next-generation drugs/technologies is crucial due to side effects, resistance, and response issues with existing drugs.

Target Protein Degradation Technology (TPD) is known as a new strategy to overcome the challenges in drug development due to the limitations of existing drug development technologies focusing on inhibiting the function of disease-related proteins. Targeted protein degradation technology is a novel concept of selectively degrading proteins, essentially removing disease-related proteins chemically. Chemically induced target protein degradation is a novel method for drug development of low molecular compounds. Low molecular compounds in TPD are used to promote the interaction between components of various cellular protein degradation pathways and target protein(s), inducing the degradation of target protein(s) as a treatment method. TPD can be applied to diseases with drug targets that were previously undruggable, essentially eliminating disease-causing proteins and potentially overcoming side effects and resistance of existing drugs.

In the field of TPD, proteolysis-targeting chimera (PROTAC), which utilizes E3 ligase, is a platform that uses small molecules that induce specific protein degradation. PROTAC molecules are small molecules with dual functions, binding both the target protein and E3-ubiquitin ligase, leading to polyubiquitin action and subsequent degradation of the target protein by the proteasome. PROTAC molecules act to target cellular proteins with good tissue distribution and ability. However, PROTAC molecules have the issue of resistance through E3 ligase mutation. Therefore, the PROTAC technology requires the discovery of new suitable E3 ligases considering substrate specificity for new target proteins.

On the other hand, the CMPD technology of the present invention promotes and induces protein degradation through the Ubiquitin-Proteasome System (UPS pathway) through interactions with various E3 ligases in vivo mediated by the chaperone complex without the direct involvement of E3 ligase. This can potentially overcome the limitations of the existing PROTAC technology.

Moreover, if the disease-related target protein is a client protein of the chaperone complex, it can be selectively targeted for degradation. This approach allows for a broader range of targets compared to PROTAC, offering higher scalability. Such versatility enables extensive research across various diseases, including cancer, degenerative brain diseases, and rare diseases, and enhances the potential for pipeline expansion.

Lung cancer is a prevalent type of cancer worldwide and a leading cause of cancer-related deaths. While there have been improvements in the 5-year survival rate since the 2000s, it still remains low at 28.2%. In cases with distant metastasis, particularly in Korea, the observed survival rate is even lower, at 6.1%. Non-small cell lung cancer (NSCLC), with various genetic mutations, has a significant unmet need despite multiple developed treatments. Research is ongoing for treatments targeting resistance mutations and combination therapies. Among NSCLC patients, 30% are caused by epidermal growth factor receptor (EGFR) mutations. This has spurred the development and growth of the treatment market, focusing initially on first-generation EGFR inhibitors. Subsequently, second-generation treatments have emerged, specifically targeting the EGFR T790M resistance mutations, which often develop as a result of resistance to the first-generation EGFR inhibitors. Among all NSCLC patients, those who develop the disease due to mutations in the c-MET (Hepatocyte Growth Factor Receptor) gene account for about 6%, making it a relatively uncommon type. However, MET mutations, which are a primary cause of resistance following treatment with first and second-generation EGFR therapies, affect approximately 250,000 patients. This has led to increasing expectations and demand for MET-targeted therapies in the NSCLC treatment market.

The present invention involves the development of CMPD-based drugs targeting the MET protein, presenting a new alternative for the treatment of NSCLC diseases with MET mutations through the degradation of target proteins.

The present inventors have utilized various linkers in CMPD inducing compounds, which bind to proteins related to the chaperone complex. By combining these compounds with existing target drugs (e.g., crizotinib, capmatinib, tepotinib) that bind to the target proteins, the present inventors have designed the structure of a CMPD-based drug. The present inventors have successfully induced the degradation and complete elimination of MET, a key target protein in NSCLC treatments, using CMPD, demonstrating the potential for superior anticancer effects through complete degradation of MET compared to existing cancer treatments, leading to the completion of the present invention.

### DISCLOSURE OF INVENTION

### Technical Problem

The object of the present invention is to provide a compound that degrades proteins by CMPD (chaperone-mediated protein degradation).

Another object of the present invention is to provide a method for producing a compound that degrades proteins by CMPD.

Another object of the present invention is to provide a use of a compound that degrades proteins by CMPD.

### Solution to Problem

The present invention provides a compound that degrades a target protein based on CMPD technology. Specifically, the present invention provides a target protein degradation compound that includes a first moiety (Chaperone Binding, CB) capable of binding to the chaperone protein or protein components of the chaperone complex, and a second moiety (Target Binding, TB) capable of binding to the target protein or proteins.

The compound according to the present invention is represented by the following Formula I, comprising a chaperone binding part (hereinafter referred to as ‘CB’)-linker (L)-target protein binding part (hereinafter referred to as ‘TB’), or a stereoisomer thereof.

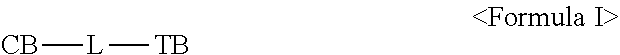

CB is the first moiety capable of binding to the chaperone protein or protein component of the chaperone complex and is either

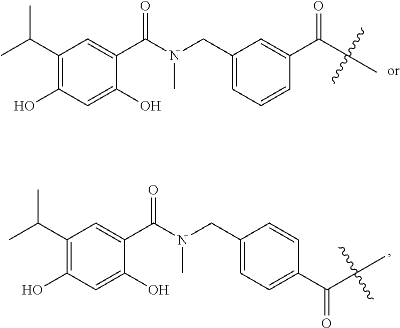 [SMILES: *C(=O)c1cccc(CN(C)C(=O)c2cc(C(C)C)c(O)cc2O)c1 ; *C(=O)c1ccc(CN(C)C(=O)c2cc(C(C)C)c(O)cc2O)cc1]

and exists in either a meta or para form.

The chaperone complex of the present invention can be composed of HSP90 (heat shock protein 90), HSP70 (heat shock protein 70), IAP (inhibitor of apoptosis protein), E3 ligase (e.g., CHIP (Carboxyl Terminus of HSC70-interacting Protein), HECTD3 (Homologous to E6AP C-Terminus domain containing 3), CUL5 (cullin 5)), and other cofactors or cochaperones.

TB, a second moiety capable of binding to the target protein or proteins, is selected from the group consisting of

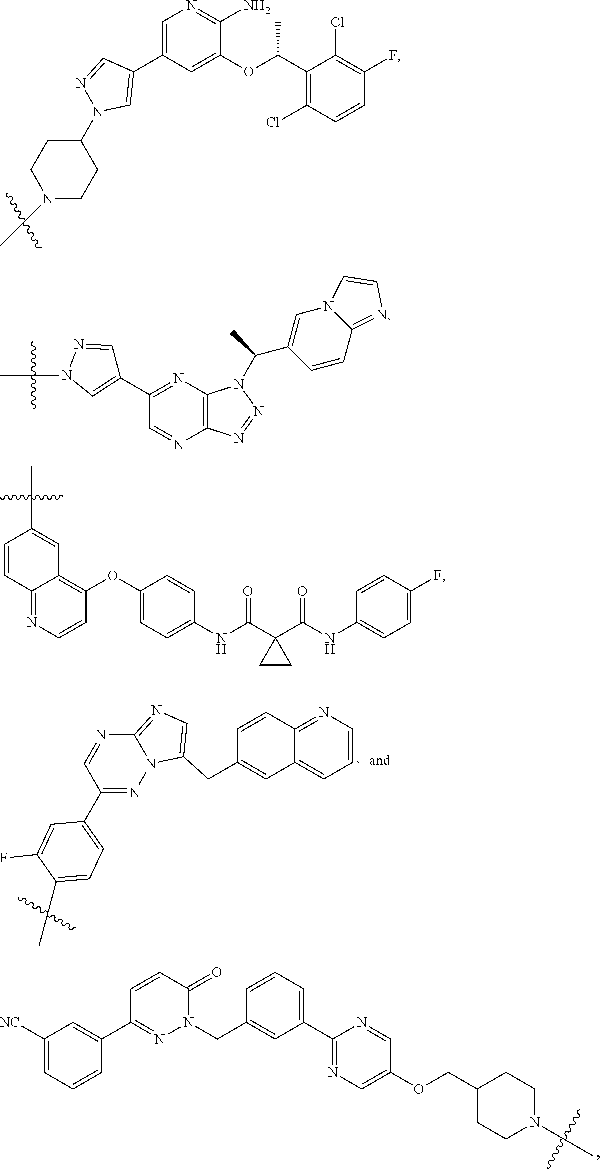 [SMILES: *N1CCC(n2cc(-c3cnc(N)c(O[C@H](C)c4c(Cl)ccc(F)c4Cl)c3)cn2)CC1 ; *n1cc(-c2cnc3nnn([C@@H](C)c4ccc5nccn5c4)c3n2)cn1 ; *c1ccc2nccc(Oc3ccc(NC(=O)C4(C(=O)Nc5ccc(F)cc5)CC4)cc3)c2c1 ; *c1ccc(-c2cnc3ncc(Cc4ccc5ncccc5c4)n3n2)cc1F ; *N1CCC(COc2cnc(-c3cccc(Cn4nc(-c5cccc(C#N)c5)ccc4=O)c3)nc2)CC1]

and preferably is selected from

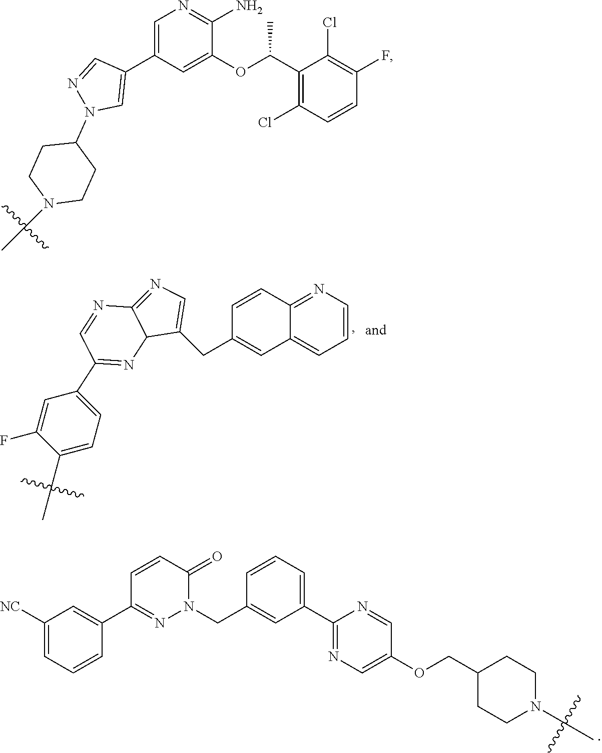 [SMILES: *N1CCC(n2cc(-c3cnc(N)c(O[C@H](C)c4c(Cl)ccc(F)c4Cl)c3)cn2)CC1 ; *c1ccc(C2=NC3C(Cc4ccc5ncccc5c4)=CN=C3N=C2)cc1F ; *N1CCC(COc2cnc(-c3cccc(Cn4nc(-c5cccc(C#N)c5)ccc4=O)c3)nc2)CC1]

The target protein according to the present invention may be selected from target drugs such as crizotinib, savolitinib, cabozantinib, capmatinib, and tepotinib.

L is a linker that binds the first moiety (CB) capable of binding to the chaperone protein or protein component of the chaperone complex and the second moiety (TB) capable of binding to a target protein or proteins, and is selected from the group consisting of

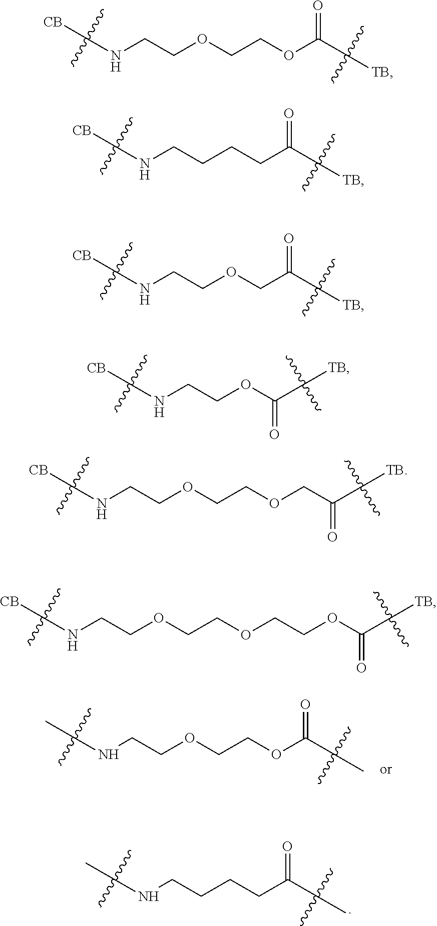

and preferably is

In the present invention, the linker is non-degradable in vivo and does not interfere with the ability of each of its binding components, which are the chaperone complex-related protein binding substance (Chaperone Binding) and target protein binding substance (Target Binding).

The present invention can produce the compound represented by Formula I having the structures listed in Table 1 below.

TABLE 1

| Com- | |
|---|---|
| pound | Structure |
| OZD- MET 01 | 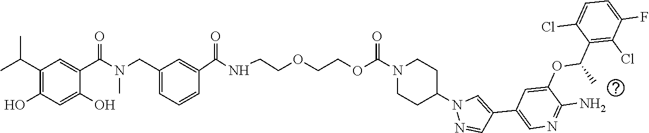 [SMILES: CC(C)c1cc(C(=O)N(C)Cc2cccc(C(=O)NCCOCCOC(=O)N3CCC(n4cc(-c5cnc(N)c(O[C@@H](C)c6c(Cl)ccc(F)c6Cl)c5)cn4)CC3)c2)c(O)cc1O] |
| OZD- MET 02 | 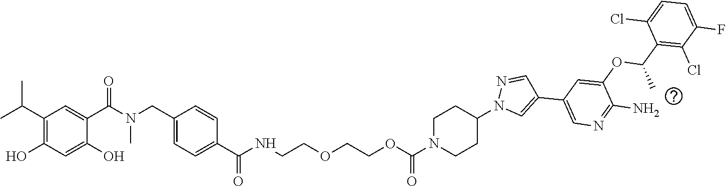 [SMILES: CC(C)c1cc(C(=O)N(C)Cc2ccc(C(=O)NCCOCCOC(=O)N3CCC(n4cc(-c5cnc(N)c(O[C@@H](C)c6c(Cl)ccc(F)c6Cl)c5)cn4)CC3)cc2)c(O)cc1O] |
| OZD- MET 03 | 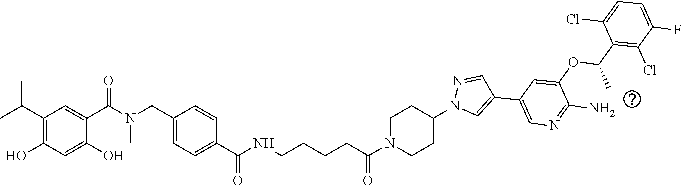 [SMILES: CC(C)c1cc(C(=O)N(C)Cc2ccc(C(=O)NCCCCC(=O)N3CCC(n4cc(-c5cnc(N)c(O[C@@H](C)c6c(Cl)ccc(F)c6Cl)c5)cn4)CC3)cc2)c(O)cc1O] |
| OZD- MET 04 | 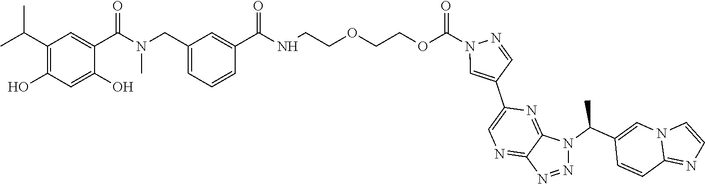 [SMILES: CC(C)c1cc(C(=O)N(C)Cc2cccc(C(=O)NCCOCCOC(=O)n3cc(-c4cnc5nnn([C@@H](C)c6ccc7nccn7c6)c5n4)cn3)c2)c(O)cc1O] |
| OZD- MET 05 | 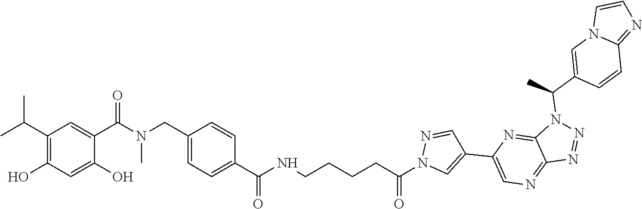 [SMILES: CC(C)c1cc(C(=O)N(C)Cc2ccc(C(=O)NCCCCC(=O)n3cc(-c4cnc5nnn([C@@H](C)c6ccc7nccn7c6)c5n4)cn3)cc2)c(O)cc1O] |
| OZD- MET 06 | 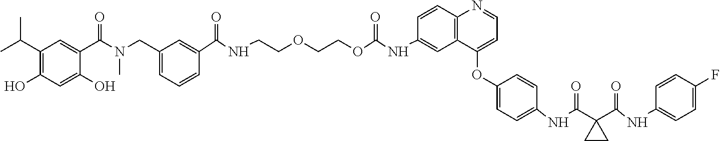 [SMILES: CC(C)c1cc(C(=O)N(C)Cc2cccc(C(=O)NCCOCCOC(=O)Nc3ccc4nccc(Oc5ccc(NC(=O)C6(C(=O)Nc7ccc(F)cc7)CC6)cc5)c4c3)c2)c(O)cc1O] |
| OZD- MET 07 | 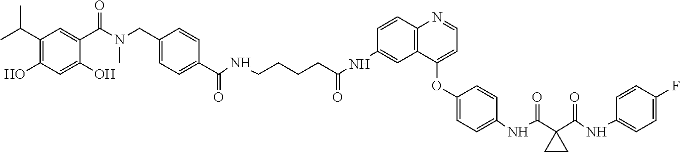 [SMILES: CC(C)c1cc(C(=O)N(C)Cc2ccc(C(=O)NCCCCC(=O)Nc3ccc4nccc(Oc5ccc(NC(=O)C6(C(=O)Nc7ccc(F)cc7)CC6)cc5)c4c3)cc2)c(O)cc1O] |
| OZD- MET 08 | 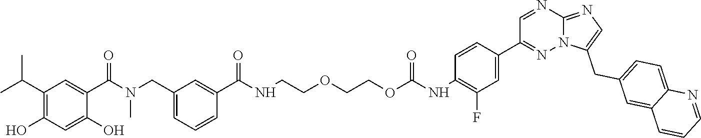 [SMILES: CC(C)c1cc(C(=O)N(C)Cc2cccc(C(=O)NCCOCCOC(=O)Nc3ccc(-c4cnc5ncc(Cc6ccc7ncccc7c6)n5n4)cc3F)c2)c(O)cc1O] |
| OZD- MET 09 | 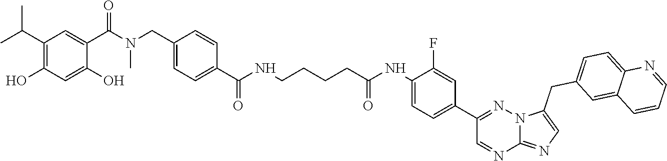 [SMILES: CC(C)c1cc(C(=O)N(C)Cc2ccc(C(=O)NCCCCC(=O)Nc3ccc(-c4cnc5ncc(Cc6ccc7ncccc7c6)n5n4)cc3F)cc2)c(O)cc1O] |
| OZD- MET 10 | 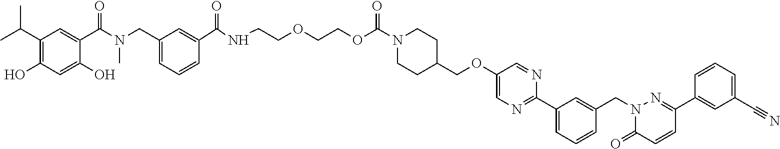 [SMILES: CC(C)c1cc(C(=O)N(C)Cc2cccc(C(=O)NCCOCCOC(=O)N3CCC(COc4cnc(-c5cccc(Cn6nc(-c7cccc(C#N)c7)ccc6=O)c5)nc4)CC3)c2)c(O)cc1O] |
| OZD- MET 11 | 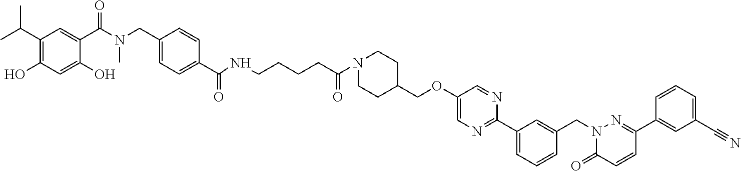 [SMILES: CC(C)c1cc(C(=O)N(C)Cc2ccc(C(=O)NCCCCC(=O)N3CCC(COc4cnc(-c5cccc(Cn6nc(-c7cccc(C#N)c7)ccc6=O)c5)nc4)CC3)cc2)c(O)cc1O] |

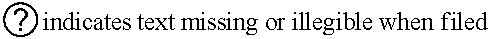

Additionally, the present invention provides a method for producing the compound represented by Formula I.

In the present invention, the compound represented by Formula I is produced through a process comprising the following steps:
i) Preparing the first moiety (CB), which can bind to the chaperone protein or protein components of the chaperone complex, specifically an acid intermediate CB in either the meta or para form;
ii) Preparing an L-TB intermediate by binding the second moiety (TB), which can bind to the target protein or proteins, with a linker (L); and
iii) Coupling the CB intermediate prepared in step i) with the L-TB intermediate prepared in step ii) to prepare the compound CB-L-TB represented by Formula I.

Step i) involves reacting Compound (a) of Formula I with Compound (b) of Formula 2 to prepare Compound (c) of Formula 3, followed by hydration to prepare the intermediate Compound (d) of Formula 4, or reacting Compound (g) of Formula 5 with Compound (b) of Formula 2 to prepare Compound (h) of Formula 6, followed by hydration to prepare the intermediate Compound (i) of Formula 7.

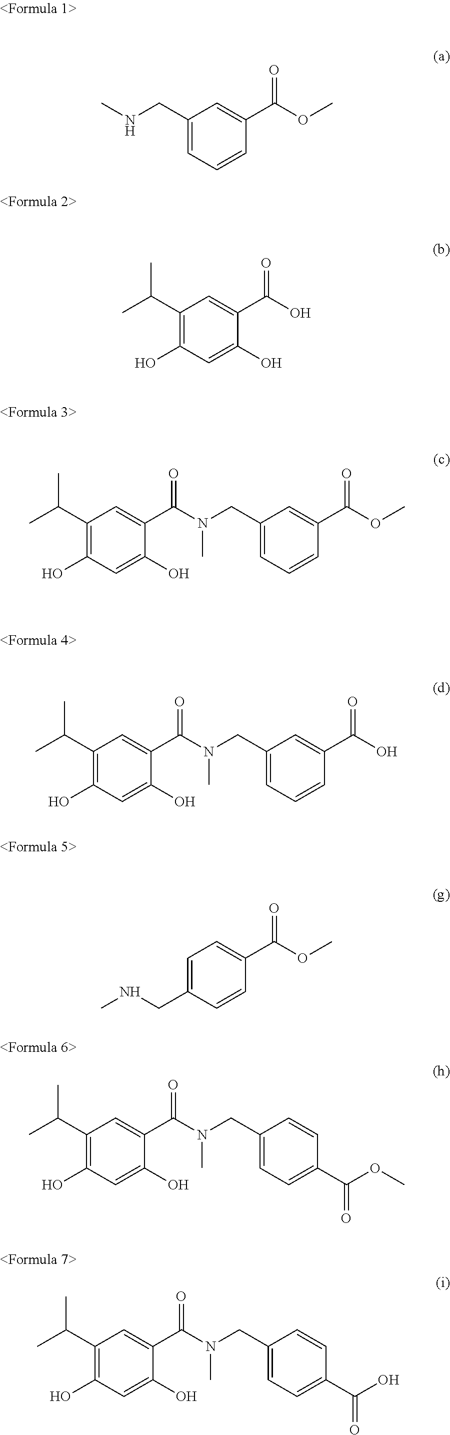

Step ii) involves reacting the target drug, which is either

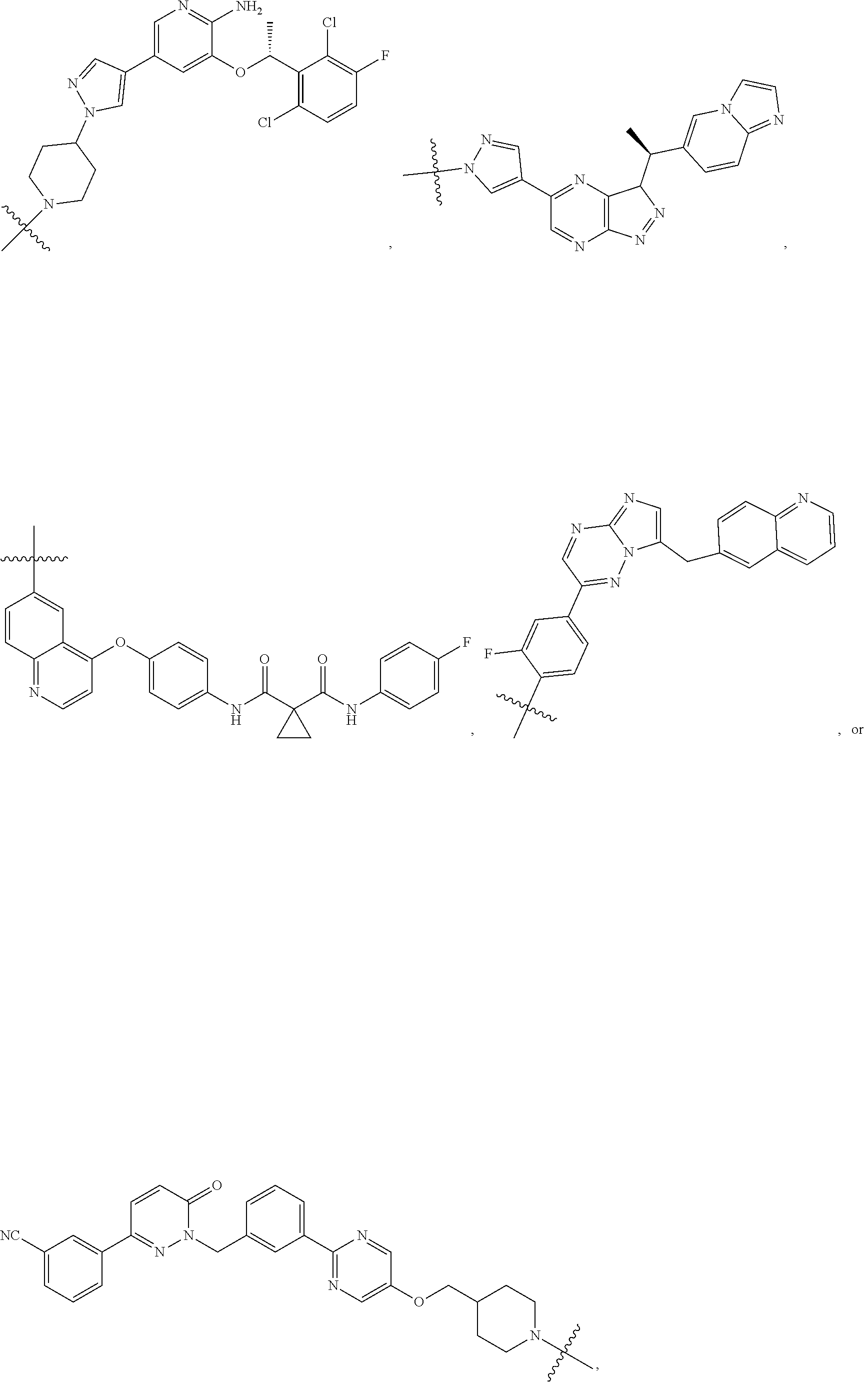

with a linker, which is either

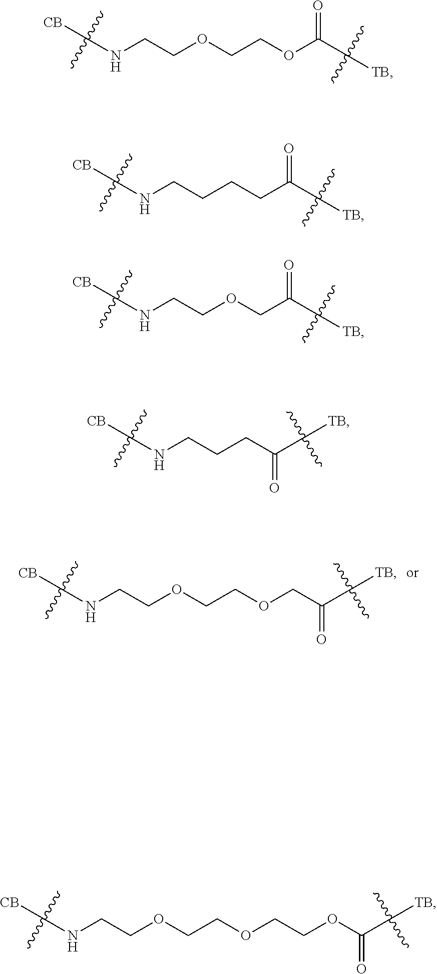

to produce the intermediate L-TB, in which the target drug and the linker are combined.

Step iii) involves coupling the intermediate Compound (d) of Formula 4 or the intermediate Compound (i) of Formula 7, prepared in step i), with the L-TB intermediate prepared in step ii) to produce the compound CB-L-TB represented by Formula I.

Furthermore, the present invention provides the use of the compound represented by Formula I.

The compounds of Formula I according to the present invention are generally useful for anti-cancer therapy. Therefore, the compounds of the present invention are useful for the treatment of cancer. The terms “cancer” and “tumor” are well known in the art and represent, in a subject, the presence of cells with typical characteristics of cancer-causing cells, such as uncontrolled proliferation, immortality, metastatic potential, rapid growth and proliferation rates, decreased cell death/apoptosis, and certain characteristic morphological features. Cancer cells are often in the form of solid tumors. However, cancer can also include non-solid tumors, such as hematologic malignancies, e.g., leukemia, where cancer cells are derived from the bone marrow. As used herein, the term “cancer” includes premalignant as well as malignant cancers. Cancers include, but are not limited to, acoustic neuroma, acute leukemia, acute lymphoblastic leukemia, acute myeloid leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchioloalveolar carcinoma, cervical cancer, chondrosarcoma, chordoma, choriocarcinoma, chronic leukemia, chronic lymphocytic leukemia, chronic myeloid (granulocytic) leukemia, chronic myelogenous leukemia, colorectal cancer, colon cancer, craniopharyngioma, cystadenocarcinoma, diffuse large B-cell lymphoma, Burkitt lymphoma, dysproliferative changes (dysplasia and metaplasias), embryonal carcinoma, endometrial cancer, endothelial sarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatic tumor, hepatocellular carcinoma, hormone-refractory prostate cancer, leiomyosarcoma, liposarcoma, lung cancer, lymphangioendothelioma, lymphangiosarcoma, lymphoblastic leukemia, lymphoma (Hodgkin and non-Hodgkin), malignancies and hyperproliferative disorders of the bladder, breast, colon, lung, ovary, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myeloid leukemia, myeloma, myxosarcoma, neuroblastoma, non-small cell lung cancer, oligodendroglioma, oral cancer, osteosarcoma, ovarian cancer, pancreatic cancer, papillary adenocarcinoma, papillary carcinoma, pinealoma, polycythemia vera, prostate cancer, rectal cancer, renal cell carcinoma, retinoblastoma, rhabdomyosarcoma, sarcomas, sebaceous carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumor, uterine cancer, and Wilms' tumor. Other cancers include primary cancer, metastatic cancer, oropharynx cancer, hypopharyngeal cancer, liver cancer, gallbladder cancer, bile duct cancer, small intestine cancer, urinary tract cancer, kidney cancer, urothelial cancer, gynecologic cancers, uterine cancer, gestational trophoblastic disease, male genital cancers, seminal vesicle cancer, testicular cancer, germ cell tumor, endocrine tumor, thyroid cancer, adrenal cancer, pituitary cancer, hemangioma, sarcoma arising from bone and soft tissue, Kaposi's sarcoma, nerve cancer, eye cancer, meningeal cancer, glioblastoma, neuroma, neuroblastoma, schwannomas, solid tumors arising from hematopoietic malignancies such as leukemia, metastatic melanoma, recurrent or persistent ovarian epithelial cancer, fallopian tube cancer, primary peritoneal cancer, gastrointestinal stromal tumor, colon cancer, stomach cancer, melanoma, glioblastoma multiforme, non-squamous non-small cell lung cancer, malignant glioma, epithelial ovarian cancer, primary peritoneal serous cancer, metastatic liver cancer, neuroendocrine carcinoma, refractory malignancies, triple-negative breast cancer, HER2-amplified breast cancer, nasopharyngeal cancer, oral cancer, cholangiocarcinoma, hepatocellular carcinoma, squamous cell carcinoma of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, progressive metastatic cancer, solid tumor, triple negative breast cancer, colon cancer, sarcoma, melanoma, renal carcinoma, endometrial cancer, thyroid cancer, rhabdomyosarcoma, multiple myeloma, ovarian cancer, glioblastoma, gastrointestinal stromal tumor, mantle cell lymphoma, and refractory malignancies.

The pharmaceutical composition according to this invention can be formulated into various dosage forms for administration using conventional methods, including oral preparations such as powders, granules, tablets, capsules, suspensions, emulsions, syrups, and aerosols, as well as topical preparations, suppositories, and sterile solutions for injection. When formulated, the preparation is made using commonly used diluents or excipients such as fillers, bulking agents, binders, wetting agents, disintegrants, and surfactants. Solid preparations for oral administration include tablets, pills, powders, granules, capsules, and the like. These solid preparations can be prepared by mixing the compound with at least one excipient, such as starch, calcium carbonate, sucrose or lactose, gelatin, and the like. In addition to simple excipients, lubricants such as magnesium stearate and talc are also used. Liquid preparations for oral administration can include suspensions, oral solutions, emulsions, syrups, and the like. Such preparations may contain, besides commonly used simple diluents like water and liquid paraffin, various excipients such as wetting agents, sweeteners, flavoring agents, preservatives, and the like. Preparations for parenteral administration can include sterile aqueous solutions, non-aqueous solvents, suspensions, emulsions, lyophilized preparations, and suppositories. Non-aqueous solvents and suspensions can include propylene glycol, polyethylene glycol, vegetable oil such as olive oil, and injectable esters such as ethyl oleate. Bases for suppositories may include materials like witepsol, macrogol, Tween 61, cocoa butter, laurin wax, glycerogelatin, and the like. All modes of administration can be anticipated, including oral or rectal administration, as well as by intravenous, intramuscular, subcutaneous, endobronchial inhalation, intrauterine, intrathecal, or intracerebrovascular injection.

Additionally, the dosage of the pharmaceutical composition according to the present invention may be increased or decreased depending on the route of administration, severity of disease, gender, weight, age, and the like. The specific dosage and treatment regimen for a particular patient will depend on various factors including the activity of the specific compound used, age, weight, general health, gender, diet, timing of administration, rate of excretion, drug combination, and the judgment of the treating physician and the severity of the specific condition being treated. Accordingly, the dosages mentioned herein shall not limit the scope of the present invention in any way.

### Advantageous Effects of Invention

The compound of the present invention has the effect of inducing the degradation of the MET protein based on CMPD technology. Therefore, the compound of the present invention can be expected to exhibit superior anticancer effects by completely degrading the target protein (MET), thereby overcoming resistance and mutations associated with targeted anticancer drugs.

### BRIEF DESCRIPTION OF DRAWINGS

FIG. 1 shows the trend of target protein degradation over time in H596 cells by the compound OZD-MET 01 of the present invention.

FIG. 2 shows the trend of target protein degradation according to dose changes in H596 cells by the compound OZD-MET 01 of the present invention.

FIG. 3 shows the trend of target protein degradation over time in H1437 cells by the compound OZD-MET 01 of the present invention.

FIG. 4 shows the trend of target protein degradation according to dose changes in H1437 cells by the compound OZD-MET 01 of the present invention.

FIG. 5 shows the trend of target protein degradation by compounds OZD-MET 01 to 11 of the present invention in H596 cells at a dose of 5 μM after 72 hours.

FIG. 6 shows the trend of target protein degradation by compounds OZD-MET 01 to 11 of the present invention in H1437 cells at a dose of 5 μM after 72 hours.

FIG. 7 shows the reduction and recovery of target protein expression in H596 cells by the compound OZD-MET 01 of the present invention.

FIG. 8 shows the reduction and recovery of target protein expression in H1437 cells by the compound OZD-MET 01 of the present invention.

FIG. 9 shows the reduction and recovery of target protein expression in H596 cells by the compound OZD-MET 02 of the present invention.

FIG. 10 shows the reduction and recovery of target protein expression in H1437 cells by the compound OZD-MET 02 of the present invention.

FIG. 11 shows the reduction and recovery of target protein expression in H596 cells by the compound OZD-MET 03 of the present invention.

FIG. 12 shows the reduction and recovery of target protein expression in H1437 cells by the compound OZD-MET 03 of the present invention.

### BEST MODE FOR CARRYING OUT THE INVENTION

The present invention is further described below with examples and experimental examples. The following examples and experimental examples are provided for illustrative purposes only to further describe the present invention exemplarily to a skilled person and shall not be construed as limiting the scope of the present invention.

### <Example 1> Preparation of 2-{2-[(3-{[1-(2,4-dihydroxy-5-isopropylphenyl)-N-methylformamido]methyl}phenyl)formamido]ethoxy}ethyl 4-(4-{6-amino-5-[1-(2,6-dichloro-3-fluorophenyl)ethoxy]pyridin-3-yl}pyrazol-1-yl)piperidine-1-carboxylate (hereinafter referred to as ‘OZD-MET 01’)

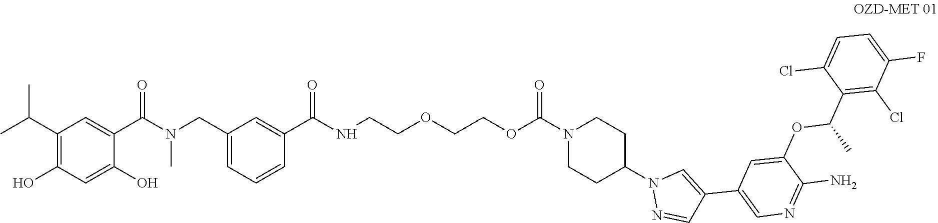 [SMILES: CC(C)c1cc(C(=O)N(C)Cc2cccc(C(=O)NCCOCCOC(=O)N3CCC(n4cc(-c5cnc(N)c(O[C@@H](C)c6c(Cl)ccc(F)c6Cl)c5)cn4)CC3)c2)c(O)cc1O]

### 1) Preparation of Intermediate Compound (d) (Meta)

Methyl 3-formylbenzoate was dissolved in methanol, and methylamine was added. Then, at 0° C., sodium borohydride was added and the resulting mixture was stirred. Once all the starting materials had reacted, methanol was removed under reduced pressure. The residue was dissolved in methylene chloride, washed with a small amount of brine (saturated sodium chloride aqueous solution), dried over MgSO₄, and filtered. The solvent was removed under reduced pressure to obtain the following compound (a), which was used immediately in the next reaction.

2,4-dihydroxy-5-isopropylbenzoate was dissolved in methanol:tetrahydrofuran (THF):H₂O (1:1:1). Lithium hydroxide was added, and the mixture was allowed to react for 12 hours. After the completion of the reaction, the mixture was filtered through Celite 545, and the solvent was removed under reduced pressure. H₂O was added to dissolve the residue, which was then acidified with hydrochloric acid. After extraction with ethyl acetate (EA) twice, washing with brine, drying over MgSO₄, filtration, and removing the solvent under reduced pressure, the following compound (b) was obtained with a yield of 97%.

Compound (a) and compound (b) were dissolved in dimethylformamide. Then, EDC (1-ethyl-(3-dimethylaminopropyl) carbodiimide), HOBT (hydroxybenzotriazole), and DIPEA (N,N-diisopropylethylamine) were added. The mixture was stirred at room temperature for 12 hours to complete the reaction. Sodium bicarbonate aqueous solution (NaHCO₃ (aq.) was added to the solution, which was then extracted twice with EA. After washing with brine and drying over MgSO₄, the mixture was filtered, and the solvent was removed under reduced pressure. The product was purified using a silica gel column to yield a clear liquid compound (c), with an 86% yield.

Compound (c) was dissolved in methanol:THF:H₂O (1:1:1), and lithium hydroxide was added. The solution was allowed to react at room temperature for 15 hours. The solvent was removed, and the residue was dissolved in H₂O and acidified with hydrochloric acid. After extraction with EA, the mixture was washed with brine and dried over MgSO₄. A solid compound (d) was obtained with a yield of 99%.

¹H NMR (500 MHz, DMSO) δ 1.24 (d, 6H), 2.81 (s, 3H), 3.19 (m, 1H), 4.39 (t, 2H), 6.41 (s, 1H), 7.40-7.61 (m, 3H), 8.19 (m, 1H), 8.46 (m, 1H), 10.07 (s, 1H)

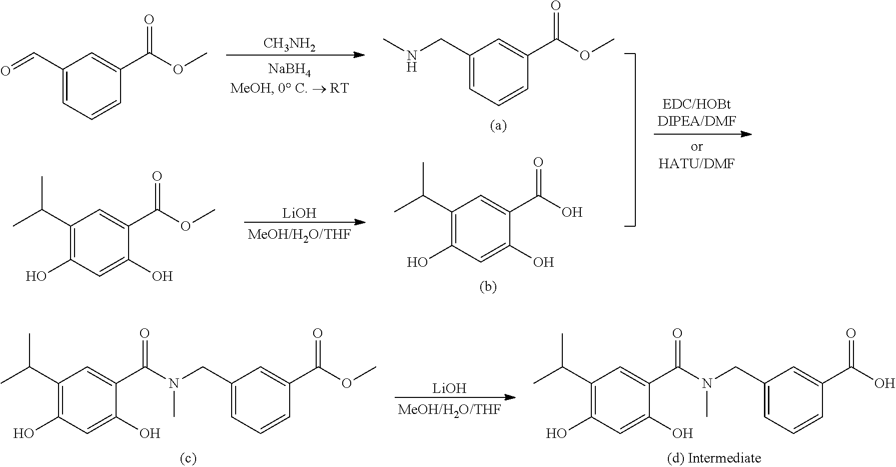

### 2) Preparation of Crizotinib-Linker Intermediate Compound (f)

Carbonyldiimidazole (CDI) was dissolved in methylene chloride, brought to 0° C., and (R)-crizotinib and 2-[2-(hydroxyethoxy)ethyl]-1H-isoindole-1,3(2H)-dione was added and the mixture was reacted for 10 hours. After all starting materials were reacted, methylene chloride was removed to obtain the following compound (e).

Compound (e) was dissolved in ethanol, and hydrazine hydrate was added, then refluxed at 110° C. for 1 hour. After stirring at room temperature, the resulting white solid was filtered to obtain the following compound (f) with a 66% yield.

¹H NMR (500 MHz, DMSO) δ 1.51-1.53 (m, 5H), 2.00 (m, 2H), 2.47 (m, 2H) 2.66 (t, 1H), 3.06-3.17 (m, 3H), 3.37 (t, 1H), 3.56-3.70 (m, 4H), 3.98 (t, 1H), 4.14-4.27 (m, 2H), 4.42 (t, 1H), 4.82 (q, 1H), 5.46 (s, 2H), 7.05 (q, 1H), 7.29 (q, 1H), 7.52-7.69 (m, 2H), 7.83 (d, 1H), 8.13 (d, 1H)

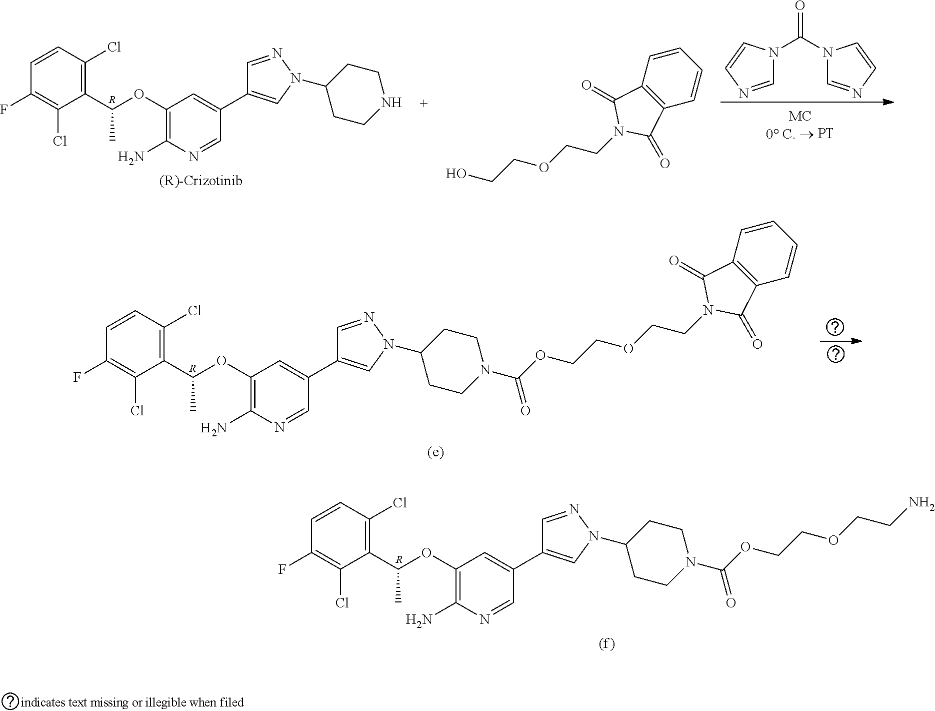

### 3) Preparation of Compound OZD-MET 01

Compound (f) and compound (d) were dissolved in DMF, to which EDC, HOBT, and DIPEA were added and the mixture was allowed to react. NaHCO₃ (aq.) was added to the solution, which was then extracted twice with EA, washed with brine, and dried over MgSO₄. The mixture was filtered, and the solvent was removed under reduced pressure. The product was purified using a silica gel column to yield a white solid compound OZD-MET 01 with a 75% yield.

¹H NMR (500 MHz, DMSO) δ 1.24 (d, 6H), 1.51 (d, 3H), 1.99 (m, 2H), 2.45 (m, 2H), 2.81 (s, 3H), 3.15-3.24 (m, 4H), 3.45-3.59 (m, 3H), 3.64-3.70 (m, 3H), 3.98 (t, 1H), 4.14 (t, 1H), 4.26-4.42 (m, 3H), 4.73-4.74 (m, 2H), 5.46 (s, 2H), 6.41 (s, 1H), 7.05-7.20 (m, 2H), 7.40-7.47 (m, 4H), 7.65-7.69 (m, 2H), 7.83-7.97 (m, 2H), 8.51-8.53 (m, 2H), 10.07 (s, 1H)

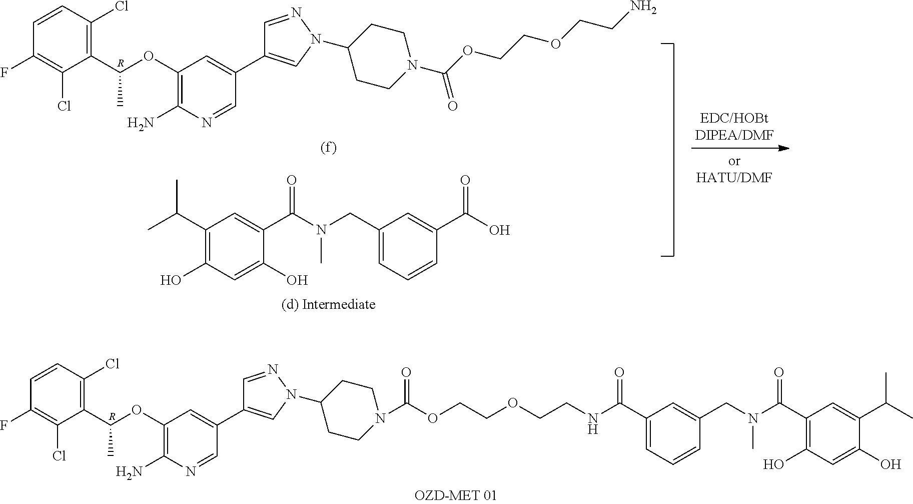

### <Example 2> Preparation of 2-{2-1 (4-{[1-(2,4-dihydroxy-5-isopropylphenyl)-N-methylformamido]methyl}phenyl)formamido]ethoxy}ethyl 4-(4-{6-amino-5-[1-(2,6-dichloro-3-fluorophenyl)ethoxy]pyridin-3-yl}pyrazol-1-yl)piperidine-1-carboxylate (Hereinafter Referred to as Compound OZD-MET 02)

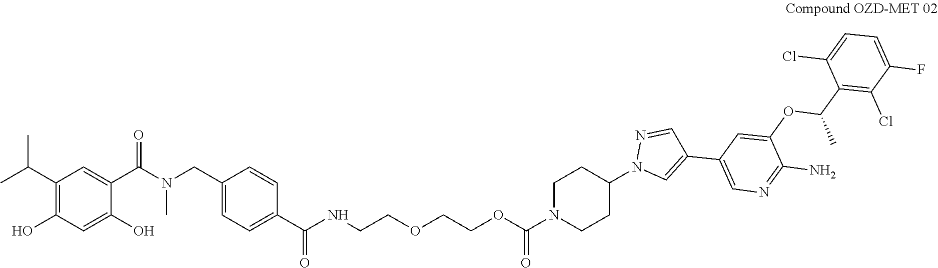 [SMILES: CC(C)c1cc(C(=O)N(C)Cc2ccc(C(=O)NCCOCCOC(=O)N3CCC(n4cc(-c5cnc(N)c(O[C@@H](C)c6c(Cl)ccc(F)c6Cl)c5)cn4)CC3)cc2)c(O)cc1O]

### 1) Preparation of Intermediate Compound (i) (Para)

Methyl 4-formylbenzoate was dissolved in methanol, and methylamine was added. Then, at 0° C., sodium borohydride was added and the mixture was stirred. After all starting materials were reacted, methanol was removed under reduced pressure. The residue was dissolved in methylene chloride, washed with H₂O (brine), dried over MgSO₄, filtered, and the solvent was removed under reduced pressure to yield the following compound (g).

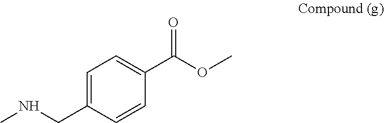

Compound (g) and compound (b) of Example 1 were dissolved in dimethylformamide. EDC, HOBT, and DIPEA were added, and the mixture was stirred at room temperature for 12 hours to complete the reaction. The solution was treated with NaHCO₃ (aq.), extracted twice with EA, washed with brine, dried over MgSO₄, filtered, and the solvent removed under reduced pressure to obtain compound (e), which was then purified using a silica gel column to yield a clear liquid compound (h) with a 52% yield.

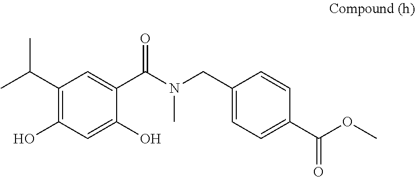 [SMILES: COC(=O)c1ccc(CN(C)C(=O)c2cc(C(C)C)c(O)cc2O)cc1]

Compound (h) was dissolved in methanol:THF:H₂O (1:1:1), and lithium hydroxide was added and allowed to react at room temperature for 15 hours. After the solvent was removed, H₂O was added to dissolve the residue. The solution was acidified with HCl, extracted with EA, washed with brine, and dried over MgSO₄ to obtain foam solid compound (i) with nearly 100% yield.

¹H NMR (500 MHz, DMSO) δ 1.24 (d, 6H), 2.81 (s, 3H), 3.19 (m, 1H), 4.42 (t, 2H), 6.41 (s, 1H), 7.42-7.61 (m, 3H), 8.02 (d, 2H), 10.07 (s, 1H)

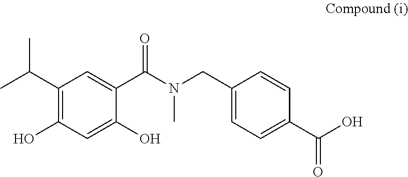 [SMILES: CC(C)c1cc(C(=O)N(C)Cc2ccc(C(=O)O)cc2)c(O)cc1O]

### 2) Preparation of Compound OZD-MET 02

The compound (i) prepared above and compound (f) prepared in 2) of Example 1 were dissolved in DMF. Then, EDC, EBOT, and DIPEA were added and the resulting mixture was allowed to react. The solution was treated with NaHCO₃ (aq.), extracted twice with EA, washed with brine, dried over MgSO₄, filtered, and the solvent removed under reduced pressure to obtain the compound. After separation using a silica gel column, a pale yellow solid compound OZD-MET 02 was obtained with a 44% yield.

¹H NMR (500 MHz, DMSO) δ 1.24 (d, 6H), 1.51 (d, 3H), 1.99 (m, 2H), 2.45 (m, 2H), 2.81 (s, 3H), 3.16-3.24 (m, 4H), 3.45-3.59 (m, 3H), 3.64-3.70 (m, 3H), 3.98 (t, 1H), 4.14 (t, 1H), 4.26-4.42 (m, 3H), 4.71-4.74 (m, 2H), 5.46 (s, 2H), 6.41 (s, 1H), 7.05-7.20 (m, 2H), 7.42-7.47 (m, 4H), 7.58-7.65 (m, 2H), 7.83 (d, 2H), 8.05 (d, 2H), 8.51 (d, 1H), 10.07 (s, 1H)

### <Example 3> Preparation of N-{[4-({5-[4-(4-{6-amino-5-[1-(2,6-dichloro-3-fluorophenyl) ethoxy]pyridine-3-yl}pyrazol-1-yl)piperidin-1-yl]-5-oxopentyl}carbamoyl)phenyl]methyl}-2,4-dihydroxy-5-isopropyl-N-methylbenzamide (Hereinafter Referred to as Compound OZD-MET 03)

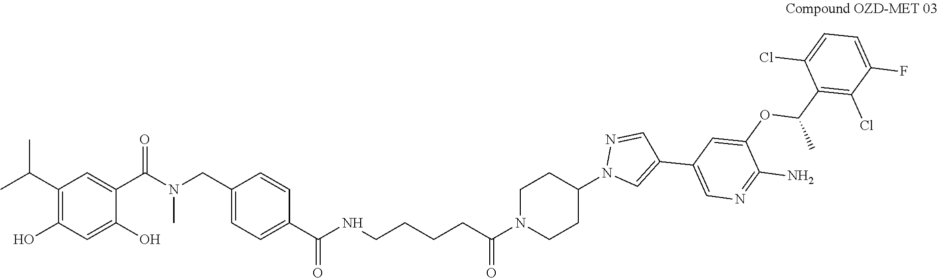 [SMILES: CC(C)c1cc(C(=O)N(C)Cc2ccc(C(=O)NCCCCC(=O)N3CCC(n4cc(-c5cnc(N)c(O[C@@H](C)c6c(Cl)ccc(F)c6Cl)c5)cn4)CC3)cc2)c(O)cc1O]

### 1) Preparation of Crizotinib-Linker Intermediate Compound (i)

(R)-crizotinib and 5-aminovaleric acid were dissolved in DMF. Then, EDC and HOBT were added and the resulting mixture was allowed to react. The solution was treated with NaHCO₃ (aq.), extracted twice with EA, washed with brine, dried over MgSO₄, filtered, and the solvent removed under reduced pressure to obtain a crude compound (j) with a 28% yield.

¹H NMR (500 MHz, DMSO) δ 1.36-1.39 (m, 3H), 1.51-1.61 (m, 5H), 1.84 (m, 1H), 1.98-1.99 (m, 3H), 2.40-2.53 (m, 4H), 3.09 (q, 2H), 3.53 (Ot, 2H), 4.29 (m, 1H), 4.77 (q, 1H), 5.46 (s, 2H), 7.05 (q, 1H), 7.30 (q, 1H), 7.51-7.61 (m, 2H), 7.83 (d, 1H), 8.13 (d, 1H)

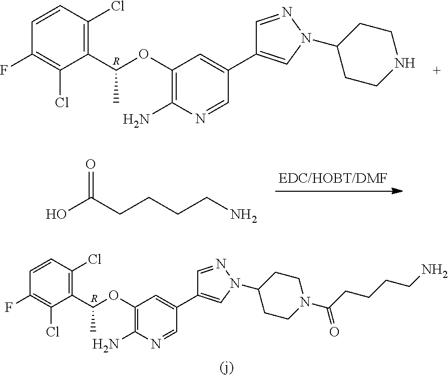

### 2) Preparation of Compound OZD-MET 03

The compound (j) prepared above and compound (i) prepared in 1) of Example 2 were dissolved in DMF. Then, EDC and HOBT were added and the resulting mixture was allowed to react. The solution was treated with NaHCO₃ (aq.), extracted twice with EA, washed with brine, dried over MgSO₄, filtered, and the solvent removed under reduced pressure to obtain the compound, which was then purified using a silica gel column to yield yellow solid compound OZD-MET 03 with a 63% yield.

¹H NMR (500 MHz, DMSO) δ 1.24 (d, 6H), 1.40 (m, 1H), 1.51 (d, 3H), 1.68 (m, 2H), 1.90-1.98 (m, 4H), 2.44-2.53 (m, 3H), 2.81 (s, 3H), 3.08 (m, 2H), 3.13-3.19 (m, 2H), 3.47-3.52 (m, 3H), 4.28-4.32 (m, 2H), 4.68-4.75 (m, 2H), 5.46 (s, 2H), 6.41 (s, 1H), 6.77 (s, 1H), 7.05 (q, 1H), 7.21 (q, 1H), 7.39-7.44 (m, 4H), 7.65-7.83 (m, 2H), 8.05 (d, 2H), 8.51 (d, 1H), 10.07 (s, 1H)

### <Example 4> Preparation of 2-{2-[(3-{[1-[2,4-dihydroxy-5-isopropylphenyl]-N-methylformamido]methyl}phenyl)formamido]ethoxy}ethyl 4-[3-(1-{imidazole[1,2-a]pyridin-6-yl}ethyl)-[1,2,3]triazolo[4,5-b]pyrazin-5-yl]pyrazole-1-carboxylate (Hereinafter Referred to as Compound OZD-MET 04)

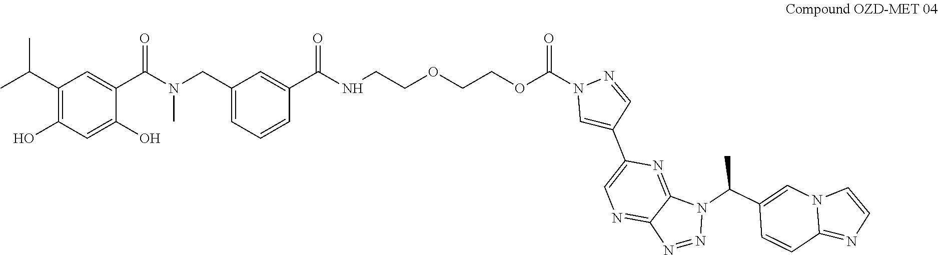 [SMILES: CC(C)c1cc(C(=O)N(C)Cc2cccc(C(=O)NCCOCCOC(=O)n3cc(-c4cnc5nnn([C@@H](C)c6ccc7nccn7c6)c5n4)cn3)c2)c(O)cc1O]

### 1) Preparation of Savolitinib-Linker Intermediate Compound (k)

Using (R)-savolitinib instead of (R)-crizotinib, following the same method as in 2) of Example 1, a savolitinib-linker intermediate compound (k) with the following structure was prepared (yield 33%).

¹H NMR (500 MHz, DMSO) δ 1.53 (s, 2H), 2.11 (d, 3H), 2.66 (t, 1H) 3.06 (t, 1H), 3.37 (t, 1H), 3.56 (t, 1H), 3.70 (t, 1H), 3.98-4.14 (m, 2H), 4.42 (t, 1H), 6.01 (m, 1H), 7.10 (d, 1H), 8.00-8.25 (m, 5H), 9.37 (m, 2H)

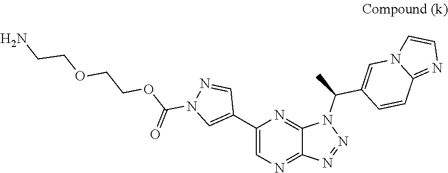 [SMILES: C[C@@H](c1ccc2nccn2c1)n1nnc2ncc(-c3cnn(C(=O)OCCOCCN)c3)nc21]

### 2) Preparation of Compound OZD-MET 04

Compound (k) and compound (d) prepared in 1) of Example 1 were dissolved in DMF. Then, EDC, HOBT, and DIPEA were added and the resulting mixture was allowed to react. The solution was treated with NaHCO₃ (aq.), extracted twice with EA, washed with brine, dried over MgSO₄, filtered, and the solvent removed under reduced pressure to obtain the compound, which was purified using a silica gel column to obtain a solid compound OZD-MET 04 with a 37% yield.

¹H NMR (500 MHz, DMSO) δ 1.24 (d, 6H), 2.10 (d, 3H), 2.81 (s, 3H), 3.19 (m, 1H), 3.41-3.45 (m, 2H), 3.64-3.70 (m, 2H), 3.98-4.14 (m, 2H), 4.42-4.46 (m, 2H), 4.89 (t, 1H), 6.00 (m, 1H), 6.41 (s, 1H), 7.40-7.42 (m, 3H), 7.69-7.85 (m, 2H), 7.97-8.17 (m, 5H), 8.57 (m, 2H), 9.37-9.44 (m, 2H), 10.07 (s, 1H)

### <Example 5> Preparation of 2,4-dihydroxy-N-({4-[(5-{4-[3-(1-{imidazole[1,2-a]pyridin-6-yl}ethyl)-[1,2,3]triazolo[4,5-b]pyrazin-5-yl]pyrazol-1-yl}-5-oxopentyl) carbamoyl]phenyl}methyl)-5-isopropyl-N-methylbenzamide (Hereinafter Referred to as Compound OZD-MET 05)

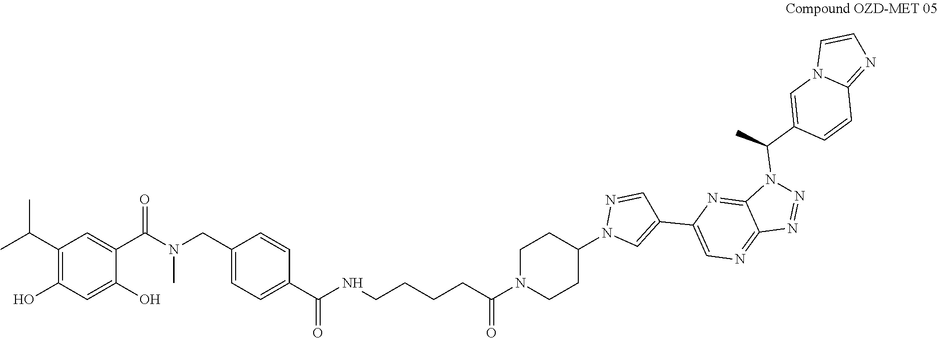 [SMILES: CC(C)c1cc(C(=O)N(C)Cc2ccc(C(=O)NCCCCC(=O)N3CCC(n4cc(-c5cnc6nnn([C@@H](C)c7ccc8nccn8c7)c6n5)cn4)CC3)cc2)c(O)cc1O]

### 1) Preparation of Savolitinib-Linker Intermediate Compound (1)

Using (R)-savolitinib instead of (R)-crizotinib, following the same method as in 1) of Example 3, a savolitinib-linker intermediate compound (1) with the following structure was prepared (yield 39%).

¹H NMR (500 MHz, DMSO) δ 1.36-1.39 (m, 3H), 1.59 (m, 1H), 1.91 (m, 1H), 2.14-2.15 (m, 4H), 2.40 (t, 1H), 2.71-2.79 (m, 2H), 3.27 (t, 1H), 6.05 (m, 1H), 7.11 (d, 1H), 7.96 (d, 1H), 8.03-8.16 (m, 4H), 9.33-9.37 (m, 2H)

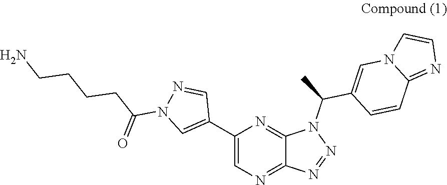 [SMILES: C[C@@H](c1ccc2nccn2c1)n1nnc2ncc(-c3cnn(C(=O)CCCCN)c3)nc21]

### 2) Preparation of Compound OZD-MET 05

Compound (1) and compound (i) prepared in 1) of Example 2 were dissolved in DMF. Then, EDC and HOBT were added and the resulting mixture was allowed to react. The solution was treated with NaHCO₃ (aq.), extracted twice with EA, washed with brine, dried over MgSO₄, filtered, and the solvent removed under reduced pressure to obtain the compound, which was purified using a silica gel column to obtain a white solid compound OZD-MET 05 with a 29% yield.

¹H NMR (500 MHz, DMSO) δ 1.24 (d, 6H), 1.39 (m, 1H), 1.70 (m, 1H), 1.97-2.20 (m, 5H), 2.71 (t, 1H), 2.81 (s, 3H), 3.13-3.27 (m, 3H), 3.47 (t, 1H), 4.48 (t, 1H), 4.91 (t, 1H), 6.03 (m, 1H), 6.41 (s, 1H), 6.77 (s, 1H), 7.42-7.68 (m, 4H), 8.02-8.17 (m, 6H), 8.54 (d, 1H), 9.37-9.39 (m, 2H), 10.01 (s, 1H)

### <Example 6> Preparation of 2-{2-[(3-{[1-(2,4-dihydroxy-5-isopropylphenyl)-N-methylformamido]methyl}phenyl)formamido]ethoxy}ethyl N-[4-(4-{1-[(4-fluorophenyl)carbamoyl]cyclopropanamido}phenoxy)quinolin-6-yl)carbamate (Hereinafter Referred to as Compound OZD-MET 06)

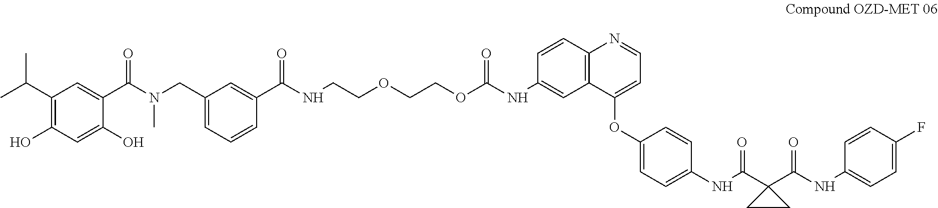 [SMILES: CC(C)c1cc(C(=O)N(C)Cc2cccc(C(=O)NCCOCCOC(=O)Nc3ccc4nccc(Oc5ccc(NC(=O)C6(C(=O)Nc7ccc(F)cc7)CC6)cc5)c4c3)c2)c(O)cc1O]

### 1) Preparation of Cabozantinib-Linker Intermediate Compound (m)

Using cabozantinib instead of (R)-crizotinib, following the same method as in 2) of Example 1, a cabozantinib-linker intermediate compound (m) with the following structure was prepared (yield 56%).

¹H NMR (500 MHz, DMSO) δ 1.49-1.53 (m, 4H), 1.72 (q, 2H), 2.86 (t, 2H), 3.46 (t, 2H), 3.84 (t, 2H), 4.28 (t, 2H), 6.65 (d, 1H), 6.95 (d, 2H), 7.20-7.45 (m, 5H), 7.71 (d, 1H), 8.20 (m, 3H), 8.59 (s, 1H), 8.82 (d, 1H), 9.83 (s, 1H), 9.92 (s, 1H)

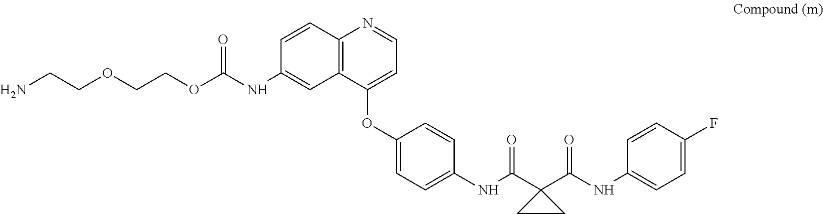 [SMILES: NCCOCCOC(=O)Nc1ccc2nccc(Oc3ccc(NC(=O)C4(C(=O)Nc5ccc(F)cc5)CC4)cc3)c2c1]

### 2) Preparation of Compound OZD-MET 06

Compound (m) and compound (d) prepared in 1) of Example 1 were dissolved in DMF. Then, EDC and HOBT were added and the resulting mixture was allowed to react. The solution was treated with NaHCO₃ (aq.), extracted three times with EA, washed with brine, dried over MgSO₄, filtered, and the solvent removed under reduced pressure to obtain the compound, which was purified using a silica gel column to obtain a dark yellow solid compound OZD-MET 06 with a yield of 42%.

¹H NMR (500 MHz, DMSO) δ 1.24 (d, 6H), 1.48 (q, 2H), 1.71 (1, 2H), 2.81 (s, 3H), 3.19-3.25 (m, 3H), 3.55 (5, 2H), 3.84 (t, 2H), 4.28-4.32 (m, 4H), 6.41 (s, 1H), 6.65 (d, 1H), 6.95 (d, 2H), 7.20-7.33 (m, 4H), 7.40-7.45 (m, 4H), 7.64-7.71 (m, 2H), 7.97 (m, 1H), 8.20-8.27 (m, 3H), 8.44-8.59 (m, 2H), 8.82 (d, 1H), 9.43-9.52 (m, 2H), 10.07 (s, 1H)

### <Example 7> Preparation of N′1-{4-[(6-{5-[(4-{[1-(2,4-dihydroxy-5-isopropylphenyl)-N-methylformamido]methyl}phenyl)formamido]pentanamido}quinolin-4-yl)oxy] phenyl}-N1-(4-fluorophenyl)cyclopropane-1,1-dicarboxamide (Hereinafter Referred to as Compound OZD-MET 07)

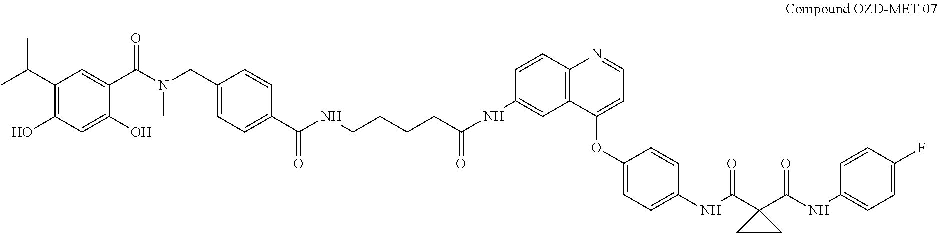 [SMILES: CC(C)c1cc(C(=O)N(C)Cc2ccc(C(=O)NCCCCC(=O)Nc3ccc4nccc(Oc5ccc(NC(=O)C6(C(=O)Nc7ccc(F)cc7)CC6)cc5)c4c3)cc2)c(O)cc1O]

### 1) Preparation of Cabozantinib-Linker Intermediate Compound (n)

Using cabozantinib instead of (R)-crizotinib, following the same method as in 1) of Example 3, a cabozantinib-linker intermediate compound (n) with the following structure was prepared (yield 63%).

¹H NMR (500 MHz, DMSO) δ 1.39 (s, 2H), 1.47-1.57 (m, 6H), 1.76 (q, 2H), 2.29 (t, 2H), 2.59 (t, 2H), 6.65 (d, 1H), 6.95 (d, 2H), 7.20-7.45 (m, 5H), 7.71 (d, 1H), 8.04-8.20 (m, 3H), 8.49 (s, 1H), 8.82 (d, 1H), 10.05 (s, 1H), 10.14 (s, 1H)

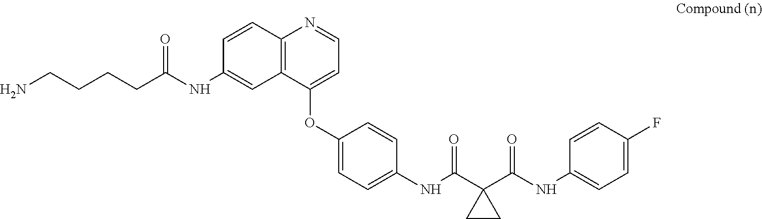 [SMILES: NCCCCC(=O)Nc1ccc2nccc(Oc3ccc(NC(=O)C4(C(=O)Nc5ccc(F)cc5)CC4)cc3)c2c1]

### 5) Preparation of Compound OZD-MET 07

Compound (n) and compound (i) prepared in 1) of Example 2 were dissolved in DMF. Then, EDC and HOBT were added and the resulting mixture was allowed to react. The solution was treated with NaHCO₃ (aq.), extracted twice with EA, washed with brine, dried over MgSO₄, filtered, and the solvent removed under reduced pressure to obtain the compound, which was purified using a silica gel column to obtain a solid compound OZD-MET 07 with a yield of 54%.

¹H NMR (500 MHz, DMSO) δ 1.24 (d, 6H), 1.51-1.54 (m, 4H), 1.63-1.74 (m, 4H), 2.29 (t, 2H), 2.81 (s, 3H), 3.19-3.30 (m, 3H), 4.34 (t, 2H), 6.41 (s, 1H), 6.65-6.77 (m, 2H), 6.95 (d, 2H), 7.20 (q, 2H), 7.33-7.45 (m, 5H), 7.63-7.71 (m, 2H), 8.05-8.20 (m, 6H), 8.82 (d, 1H), 9.65 (s, 1H), 9.75 (s, 1H), 10.07 (s, 1H)

### <Example 8> Preparation of 2-{2-[(3-{[1-(2,4-dihydroxy-5-isopropylphenyl)-N-methylformamido]methyl}phenyl)formamido]ethoxy}ethyl N-{2-fluoro-4-[7-(quinolin-6-ylmethyl)imidazo[1,2-b][1,2,4]triazin-2-yl]phenyl}carbamate (Hereinafter Referred to as Compound OZD-MET 08)

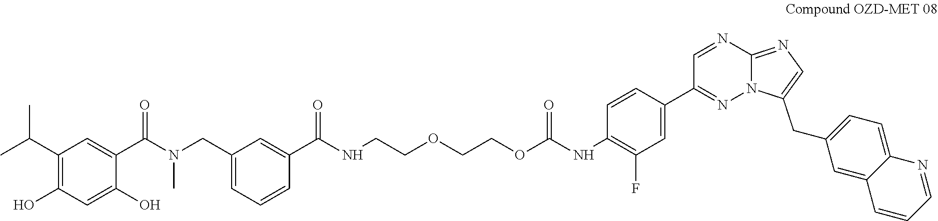 [SMILES: CC(C)c1cc(C(=O)N(C)Cc2cccc(C(=O)NCCOCCOC(=O)Nc3ccc(-c4cnc5ncc(Cc6ccc7ncccc7c6)n5n4)cc3F)c2)c(O)cc1O]

### 1) Preparation of Capmatinib-Linker Intermediate Compound (o)

Using capmatinib instead of (R)-crizotinib, following the same method as in 2) of Example 1, a capmatinib-linker intermediate compound (o) with the following structure was prepared (yield 58%).

¹H NMR (500 MHz, DMSO) δ 1.53 (s, 2H), 2.86 (t, 2H), 3.46 (t, 2H), 3.84 (t, 2H), 4.24-4.28 (m, 4H), 7.19 (m, 1H), 7.39-7.62 (m, 4H), 7.91-8.04 (m, 2H), 8.20-8.39 (m, 3H), 8.87 (q, 1H), 9.19 (s, 1H)

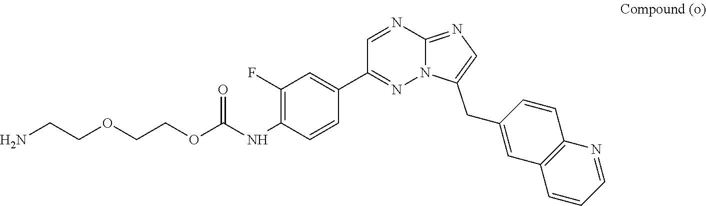 [SMILES: NCCOCCOC(=O)Nc1ccc(-c2cnc3ncc(Cc4ccc5ncccc5c4)n3n2)cc1F]

### 2) Preparation of Compound OZD-MET 08

Compound (o) and compound (d) prepared in 1) of Example 1 were dissolved in DMF. Then, EDC and HOBT were added and the resulting mixture was allowed to react. The solution was treated with NaHCO₃ (aq.), extracted twice with EA, washed with brine, dried over MgSO₄, filtered, and the solvent removed under reduced pressure to obtain the compound, which was purified using a silica gel column to obtain a solid compound OZD-MET 08 with a yield of 55%.

¹H NMR (500 MHz, DMSO) δ 1.24 (d, 6H), 2.81 (s, 3H), 3.19 (m, 1H), 3.46-3.55 (m, 4H), 3.84 (t, 2H), 4.22-4.28 (m, 4H), 4.53 (t, 2H), 6.41 (s, 1H), 7.19 (m, 1H), 7.40-7.42 (m, 3H), 7.52-7.57 (m, 2H), 7.62-7.72 (m, 4H), 7.97 (m, 1H), 8.12-8.21 (m, 2H), 8.34 (t, 1H), 8.44-8.46 (m, 2H), 8.87 (q, 1H), 9.01 (s, 1H), 10.01 (s, 1H)

### <Example 9> Preparation of N-[(4-{[4-({2-fluoro-4-[7-(quinolin-6-ylmethyl)imidazo[1,2-b][1,2,4]triazin-2-yl]phenyl}carbamoyl)butyl]carbamoyl}phenyl)methyl]-2,4-dihydroxy-5-isopropyl-N-methyl-benzamide (Hereinafter Referred to as Compound OZD-MET 09)

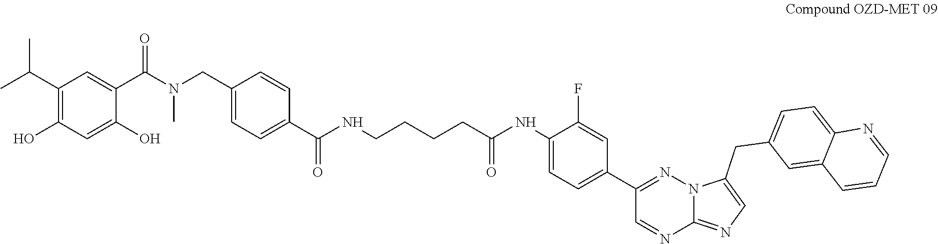 [SMILES: CC(C)c1cc(C(=O)N(C)Cc2ccc(C(=O)NCCCCC(=O)Nc3ccc(-c4cnc5ncc(Cc6ccc7ncccc7c6)n5n4)cc3F)cc2)c(O)cc1O]

### 1) Preparation of Capmatinib-Linker Intermediate Compound (p)

Using capmatinib instead of (R)-crizotinib, following the same method as in 1) of Example 3, a capmatinib-linker intermediate compound (p) with the following structure was prepared (yield 60%).

¹H NMR (500 MHz, DMSO) δ 1.39 (s, 2H), 1.47 (m, 2H), 1.77 (m, 2H), 2.29 (t, 2H), 2.59 (t, 2H), 4.27 (q, 2H), 7.19 (m, 1H), 7.39-7.44 (m, 3H), 7.62 (d, 1H), 7.91 (q, 1H), 8.21-8.40 (m, 3H), 8.64 (s, 1H), 8.87 (q, 1H), 9.19 (s, 1H)

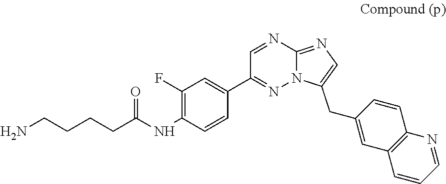 [SMILES: NCCCCC(=O)Nc1ccc(-c2cnc3ncc(Cc4ccc5ncccc5c4)n3n2)cc1F]

### 2) Preparation of Compound OZD-MET 09

Compound (p) and compound (i) prepared in 1) of Example 2 were dissolved in DMF. Then, EDC and HOBT were added and the resulting mixture was allowed to react. The solution was treated with NaHCO₃ (aq.), extracted three times with EA, washed with brine, dried over MgSO₄, filtered, and the solvent removed under reduced pressure to obtain the compound, which was purified using a silica gel column to obtain OZD-MET 09, a white solid compound, with a yield of 63%.

¹H NMR (500 MHz, DMSO) δ 1.24 (d, 6H), 1.54 (m, 2H), 1.84 (m, 2H), 2.29 (t, 2H), 2.81 (s, 3H), 3.19-3.30 (m, 3H), 4.26 (q, 2H), 4.55 (t, 2H), 6.41 (s, 1H), 6.77 (s, 1H), 7.19 (m, 1H), 7.42-7.44 (m, 4H), 7.62-7.63 (m, 2H), 8.05-8.12 (m, 3H), 8.21-8.25 (m, 2H), 8.41-8.54 (m, 2H), 8.87 (q, 1H), 9.01 (s, 1H), 10.07 (s, 1H)

### <Example 10> Preparation of 2-{2-[(3-{[1-(2,4-dihydroxy-5-isopropylphenyl)-N-methylformamido]methyl}phenyl)formamido]ethoxy}ethyl 4-({[2-(3-{[3-(3-cyanophenyl)-6-oxopyridazin-1-yl]methyl}phenyl)pyrimidin-5-yl]oxy}methyl)piperidine-1-carboxylate (Hereinafter Referred to as Compound OZD-MET 10)

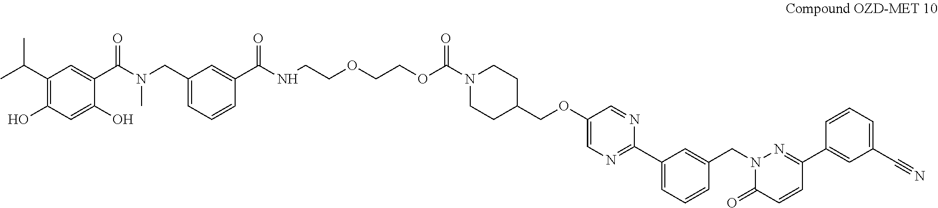 [SMILES: CC(C)c1cc(C(=O)N(C)Cc2cccc(C(=O)NCCOCCOC(=O)N3CCC(COc4cnc(-c5cccc(Cn6nc(-c7cccc(C#N)c7)ccc6=O)c5)nc4)CC3)c2)c(O)cc1O]

### 1) Preparation of Tepotinib-Linker Intermediate Compound (q)

Using tepotinib instead of (R)-crizotinib, following the same method as in 2) of Example 1, a tepotinib-linker intermediate compound (q) with the following structure was prepared (yield 76%).

¹H NMR (500 MHz, DMSO) δ 1.53 (s, 2H), 1.65 (m, 2H), 2.04 (m, 3H), 2.86 (t, 2H), 3.05 (q, 2H), 3.46-3.49 (m, 4H), 3.84 (t, 2H), 4.07 (d, 2H), 4.28 (t, 2H), 5.64 (t, 2H), 7.29-7.42 (m, 4H), 7.70-7.77 (m, 2H), 8.13-8.20 (m, 3H), 8.63 (s, 2H), 8.93 (d, 1H)

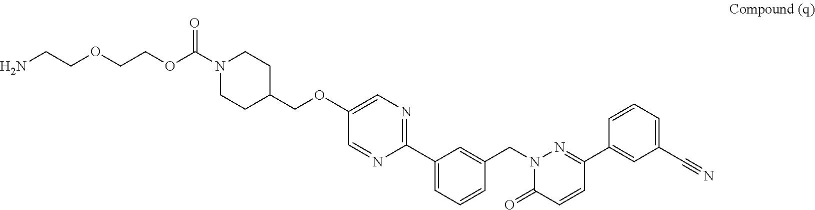 [SMILES: N#Cc1cccc(-c2ccc(=O)n(Cc3cccc(-c4ncc(OCC5CCN(C(=O)OCCOCCN)CC5)cn4)c3)n2)c1]

### 2) Preparation of Compound OZD-MET 10

Compound (q) and compound (d) prepared in 1) of Example 1 were dissolved in DMF. Then, EDC and HOBT were added and the resulting mixture was allowed to react. The solution was treated with NaHCO₃ (aq.), extracted twice with EA, washed with brine, dried over MgSO₄, filtered, and the solvent removed under reduced pressure to obtain the compound, which was purified using a silica gel column to obtain a white compound OZD-MET 10 with a yield of 78%.

¹H NMR (500 MHz, DMSO) δ 1.24 (d, 6H), 1.63 (m, 2H), 2.03 (m, 3H), 2.81 (s, 3H), 3.03-3.19 (m, 3H), 3.45-3.47 (m, 4H), 3.55 (t, 2H), 3.84 (t, 2H), 4.08-4.28 (m, 4H), 4.53 (t, 2H), 5.63 (t, 2H), 6.41 (s, 1H), 7.01 (m, 1H), 7.29-7.42 (m, 5H), 7.65-7.77 (m, 4H), 7.97 (m, 1H), 8.20 (m, 1H), 8.34-8.45 (m, 3H), 8.63 (s, 2H), 10.07 (s, 1H)

### <Example 11> Preparation of N-{[4-({5-[4-({[2-(3-{[3-(3-cyanophenyl)-6-oxopyridazin-1-yl]methyl} phenyl)pyrimidin-5-yl]oxy}methyl)piperidin-1-yl]-5-oxopentyl}carbamoyl)phenyl]methyl}-2,4-dihydroxy-5-isopropyl-N-methylbenzamide (Hereinafter Referred to as Compound OZD-MET 11)

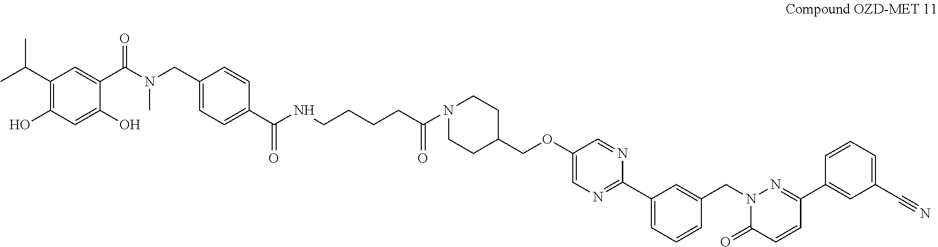 [SMILES: CC(C)c1cc(C(=O)N(C)Cc2ccc(C(=O)NCCCCC(=O)N3CCC(COc4cnc(-c5cccc(Cn6nc(-c7cccc(C#N)c7)ccc6=O)c5)nc4)CC3)cc2)c(O)cc1O]

### 1) Preparation of Tepotinib-Linker Intermediate Compound (r)

Using tepotinib instead of (R)-crizotinib, following the same method as in 1) of Example 3, a tepotinib-linker intermediate compound (r) with the following structure was prepared (yield 74%).

¹H NMR (500 MHz, DMSO) δ 1.39 (s, 2H), 1.47 (m, 2H), 1.63-1.74 (m, 4H), 2.03-2.26 (m, 5H), 2.59 (t, 2H), 2.97 (q, 2H), 3.41 (t, 2H), 4.07 (d, 2H), 5.68 (t, 2H), 7.29-7.42 (m, 4H), 7.70-7.77 (m, 2H), 8.13-8.20 (m, 3H), 8.63 (s, 2H), 8.93 (d, 1H)

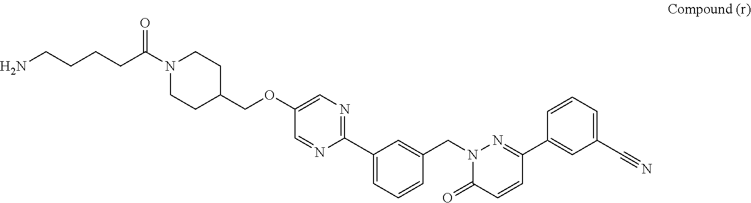 [SMILES: N#Cc1cccc(-c2ccc(=O)n(Cc3cccc(-c4ncc(OCC5CCN(C(=O)CCCCN)CC5)cn4)c3)n2)c1]

### 2) Preparation of Compound OZD-MET 11

Compound (r) and compound (i) prepared in 1) of Example 2 were dissolved in DMF. Then, EDC and HOBT were added and the resulting mixture was allowed to react. The solution was treated with NaHCO₃ (aq.), extracted twice with EA, washed with brine, dried over MgSO₄, filtered, and the solvent removed under reduced pressure to obtain the compound, which was purified using a silica gel column to obtain OZD-MET 11, a yellow solid compound, with a yield of 79%.

¹H NMR (500 MHz, DMSO) δ 1.24 (d, 6H), 1.54-1.62 (m, 4H), 1.80 (m, 2H), 2.01-2.05 (m, 3H), 2.26 (t, 2H), 2.96 (q, 2H), 3.19 (m, 1H), 3.30-3.40 (m, 4H), 4.08 (d, 2H), 4.55 (t, 2H), 5.67 (t, 2H), 6.41 (s, 1H), 6.77 (s, 1H), 7.01 (m, 1H), 7.29-7.42 (m, 5H), 7.65-7.77 (m, 3H), 8.05 (d, 2H), 8.20-8.40 (m, 3H), 9.43 (d, 1H), 10.07 (s, 1H)

### <Experimental Example 1> Cell Viability Effect

This experiment involves treating cells with each of the compounds prepared in Examples 1 to 11, and then observing changes in cell cytotoxicity and cell count. The IC₅₀ values, derived from the changes in cell count, can be used to assess the cancer cell growth inhibition and anti-cancer effects of the compound of the present invention.

### <Experimental Method> Cell Viability Assay

Cells (H596 or H1437 cells) were seeded in a 96-well plate at 3×10³ cells per well and then cultured for more than 12 hours. After removing the media, each well was treated with 200 μl of the respective diluted compounds prepared in Examples 1 to 11. Three days later, the media was removed, and each well was treated with 10 μl of CCK8 reagent+90 μl of media. After 3-4 hours of reaction, the absorbance was measured using an ELISA reader at a wavelength of 450 nM. IC₅₀ values were calculated using the Graphpad prism program and are shown in Table 2.

As can be seen in Table 2, the cell viability of each compound prepared in Examples 1 to 11 was more than 10-fold higher compared to the control drug crizotinib, and the intermediate compounds (d) and (i) of the first moiety that binds to the chaperone complex.

Therefore, the compound of the present invention exhibits significantly superior anticancer efficacy compared to the first and second moiety compounds.

TABLE 2

| | Cell viability (IC₅₀, uM) | |
|---|---|---|
| Compound | H596 | H1437 |
| OZD-MET 01 | 3.07 | 3.50 |
| OZD-MET 02 | 2.59 | 4.06 |
| OZD-MET 03 | 1.97 | 2.64 |
| OZD-MET 04 | 4.18 | 4.65 |
| OZD-MET 05 | 4.72 | 5.71 |
| OZD-MET 06 | 3.38 | 4.25 |
| OZD-MET 07 | 3.43 | 3.21 |
| OZD-MET 08 | 3.08 | 4.34 |
| OZD-MET 09 | 4.18 | 3.11 |
| OZD-MET 10 | 3.19 | 3.29 |
| OZD-MET 11 | 3.99 | 3.44 |
| Crizotinib | 20.3 | 31.9 |
| Compound (d) | NAV | NAV |
| Compound (i) | NAV | NAV |
| Note | *NAV: non-available value | *NAV: non-available value |

### <Experimental Example 2> Verification of c-MET Degradation Effect

This experiment assessed the tendency and extent of degradation of the target protein c-MET (total form) in cells treated with each of the compounds prepared in Examples 1 to 11.

### <Experimental Method> Target Protein Degradation Assay

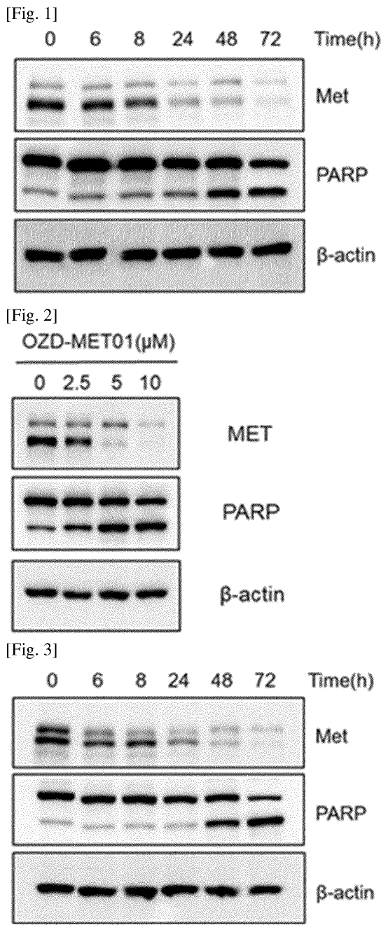

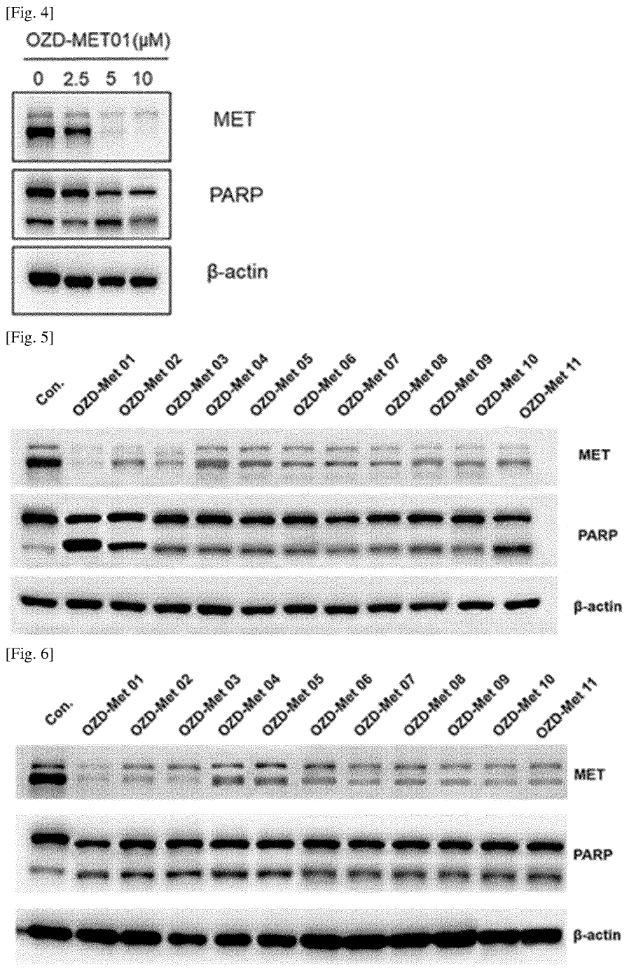

Cells (H596 or H1437 cells) were seeded in Φ60 or Φ100 culture dishes to reach 5×10⁵ or 1×10⁶ cells, and then cultured for more than 12 hours. After removing the media, the cells were treated with each respective compound prepared in Examples 1 to 11 at various conditions-concentrations (0, 2.5, 5, 10 μM), time (0, 6, 8, 24, 48, 72 hours), and standard concentration and time (5 μM, 72 hours) for all compounds. The cells were then harvested, lysed using RIPA buffer, and centrifuged (4° C., 13000 rpm, 15 min), to collect the supernatant in a new tube. Protein quantification was performed using the Bradford Assay, and samples were prepared for loading by boiling at 95° C. Western blotting was performed. The samples were loaded onto an SDS polyacrylamide gel (8% concentration). Electrophoresis was conducted at 80V, and then switched to 100-120V (when the loaded sample moves from the stacking gel to the running gel). After electrophoresis, the polyacrylamide gel was transferred onto the membrane (using transfer buffer diluted at 10× transfer buffer:methanol:D.W=1:2:7 ratio, cold, for 100V, 1 hour or 80V, 2 hours). The membrane was blocked with 5% skim milk for 30 minutes and washed with PBS-T (phosphate-buffered saline with Tween 20) buffer. The primary antibody was diluted in 5% BSA+NaN₃ solution and reacted with the membrane for over 12 hours, followed by washing with PBS-T and reaction with 5% skim milk+secondary antibody for 1 hour and 30 minutes. After washing with PBS-T, the ECL solution was reacted with the membrane, and the bands were detected using an imaging device to confirm the degradation of the target protein c-MET. The results are shown in Table 3 and FIGS. 1 to 6.

As can be seen in Table 3 below, no degradation of the target MET protein was observed with crizotinib, or the compounds (d) and (i) of the first moiety that bind to the chaperone complex. In contrast, clear degradation of the target protein was observed with each of the compounds prepared in Examples 1 to 11.

Therefore, the present invention demonstrates excellent efficacy in the degradation of target proteins.

TABLE 3

c-MET degradation

| Compound | H596 | H1437 |
|---|---|---|
| OZD-MET 01 | 1 | 1 |
| OZD-MET 02 | 1 | 1 |
| OZD-MET 03 | 1 | 1 |
| OZD-MET 04 | 2 | 3 |
| OZD-MET 05 | 2 | 2 |
| OZD-MET 06 | 2 | 2 |
| OZD-MET 07 | 1 | 2 |
| OZD-MET 08 | 1 | 2 |
| OZD-MET 09 | 2 | 1 |
| OZD-MET 10 | 1 | 1 |
| OZD-MET 11 | 2 | 1 |

| | |
|---|---|
| Crizotinib | No degradation |
| Compound (d) | No degradation |
| Compound (i) | No degradation |
| Note | *c-MET degradation(%) |
| | 50% < degradation: 1 |
| | 10% < degradation < 50%: 2 |
| | degradation < 10%: 3 |

### <Experimental Example 3> Degradation and Recovery of Target Protein Through the UPS Pathway

This experiment assessed the degradation and recovery of the target protein c-MET through the ubiquitin-proteasome system (UPS) pathway in cells treated with each compound prepared in Examples 1 to 11, in conjunction with the proteasome inhibitor bortezomib.

### <Experimental Method> Target Protein Degradation and Recovery Assay

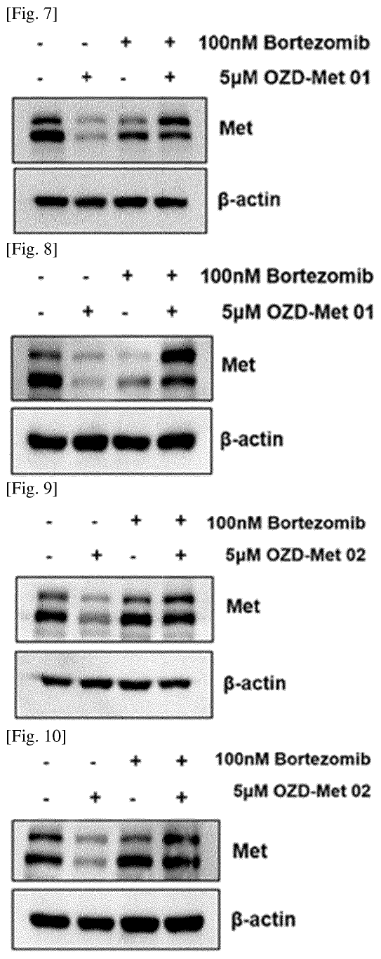

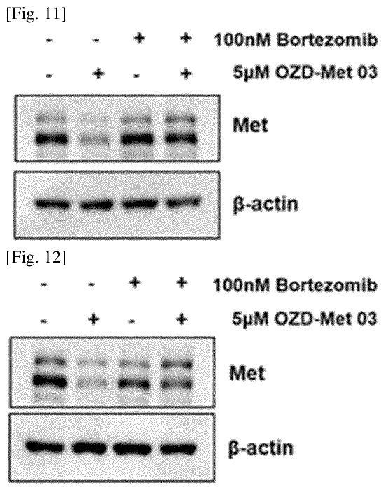

Cells (H596 or H1437 cells) were seeded in a 6-well culture plate at a density of 3×10⁵ cells, and then cultured for more than 12 hours. After replacing the media, the cells were treated with 100 nM bortezomib and 5 μM of the respective compound prepared in Examples 1 to 11. The control group was treated with the same amount of dimethylsulfoxide (DMSO), the solvent for the drug. After 16 hours, the media was removed. Cells were washed with Phosphate Buffered Saline (PBS) and harvested using trypsin-ethylenediaminetetraacetic acid (Trypsin-EDTA). Cell lysis was performed using Radio-ImmunoPrecipitation Assay buffer (RIPA buffer), followed by centrifugation (4° C., 13000 rpm, 15 min). Then, the supernatant was transferred to a new tube. Protein quantification was conducted using the Bradford Assay, followed by boiling the samples at 95° C. for 10 minutes. Western blotting was performed by loading samples onto an 8% SDS (Sodium Dodecyl Sulfate) polyacrylamide gel, running electrophoresis at 80V, and then increasing the voltage to 100V for 60 minutes at a cold temperature once the proteins moved from the stacking gel to the running gel. The membrane was blocked with 5% skim milk solution for more than 30 minutes and washed with PBS-T (phosphate-buffered saline with Tween 20) buffer. The primary antibody was diluted in 5% bovine serum albumin (5% BSA) and NaN₃ solution and reacted for more than 12 hours. After washing with PBS-T, the membrane was incubated with the secondary antibody diluted in a 5% skim milk solution and allowed to react for 1 hour and 30 minutes. The membrane was then washed with PBS-T and treated with ECL solution. Bands were detected using an imaging device. The experiment assessed whether the decrease in MET expression caused by each compound prepared in Examples 1 to 11 was recovered when treated with bortezomib. The results are presented in Table 4 and FIGS. 7 to 12.

As can be seen in Table 4, no degradation and recovery of the target protein was observed with crizotinib, or the intermediate compounds (d) and (i) of the first moiety binding to the chaperone complex. In contrast, clear degradation and recovery of the target protein was observed with each of the compounds prepared in Examples 1 to 11 when treated with bortezomib, a proteasome inhibitor.

TABLE 4

Proteasome inhibitor treatment

| Compound | H596 | H1437 |
|---|---|---|
| OZD-MET 01 | 1 | 1 |
| OZD-MET 02 | 1 | 1 |
| OZD-MET 03 | 1 | 1 |
| OZD-MET 04 | 2 | 2 |
| OZD-MET 05 | 2 | 2 |
| OZD-MET 06 | 2 | 2 |
| OZD-MET 07 | 2 | 2 |
| OZD-MET 08 | 1 | 1 |
| OZD-MET 09 | 2 | 2 |
| OZD-MET 10 | 1 | 2 |
| OZD-MET 11 | 2 | 1 |

| | |
|---|---|
| Crizotinib | No degradation & recovery |
| Compound (d) | No degradation & recovery |
| Compound (i) | No degradation & recovery |
| Note | * Recovery from degradation (%) |
| | 50% < recovery; 1 |
| | 10% < recovery < 50%; 2 |
| | recovery < 10%; 3 |

1: A compound represented by the following formula (I) or a stereoisomer thereof: 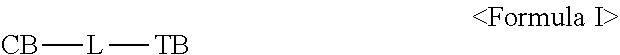
wherein
CB is either 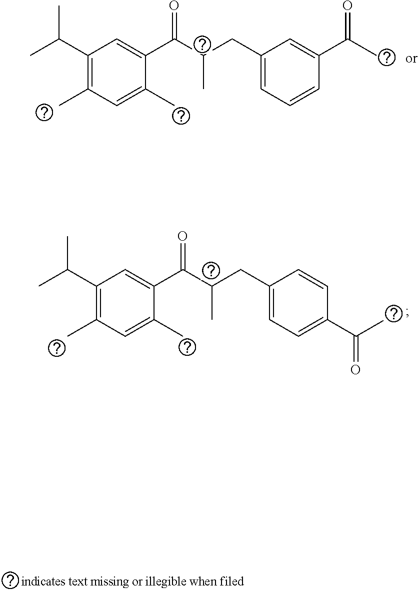
TB is one selected from the group consisting of 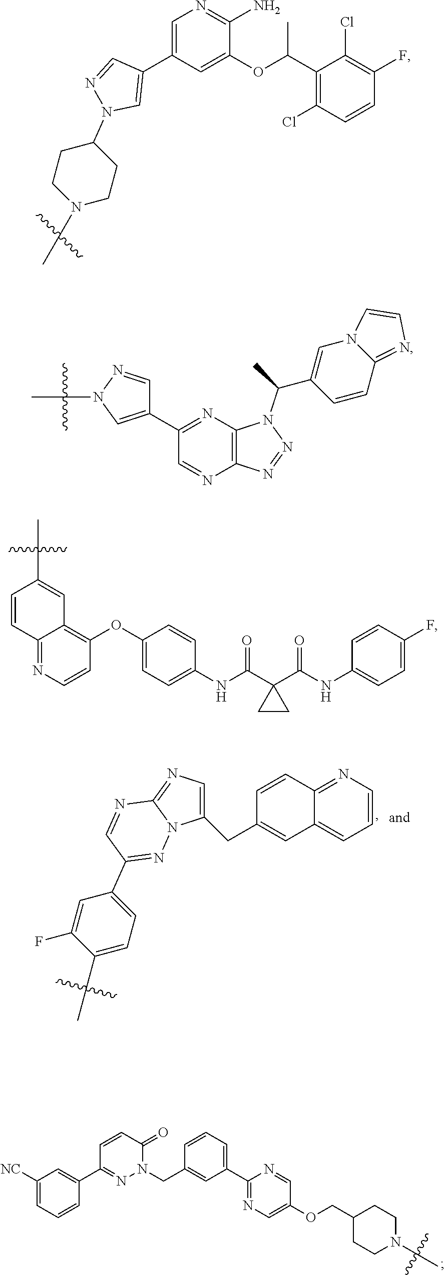 [SMILES: *N1CCC(n2cc(-c3cnc(N)c(OC(C)c4c(Cl)ccc(F)c4Cl)c3)cn2)CC1 ; *n1cc(-c2cnc3nnn([C@@H](C)c4ccc5nccn5c4)c3n2)cn1 ; *c1ccc2nccc(Oc3ccc(NC(=O)C4(C(=O)Nc5ccc(F)cc5)CC4)cc3)c2c1 ; *c1ccc(-c2cnc3ncc(Cc4ccc5ncccc5c4)n3n2)cc1F ; *N1CCC(COc2cnc(-c3cccc(Cn4nc(-c5cccc(C#N)c5)ccc4=O)c3)nc2)CC1]
L is one selected from the group consisting of

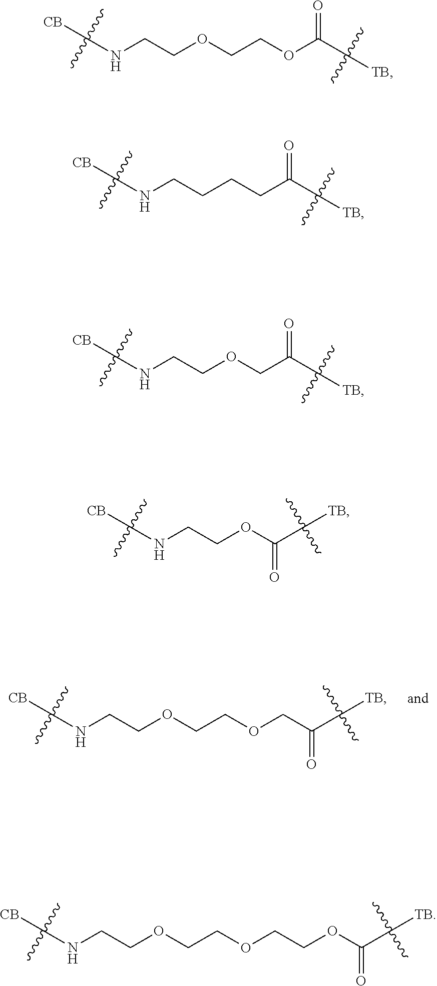

2: The compound of claim 1, wherein CB exists in meta or para form.

3: The compound of claim 1, wherein TB is either

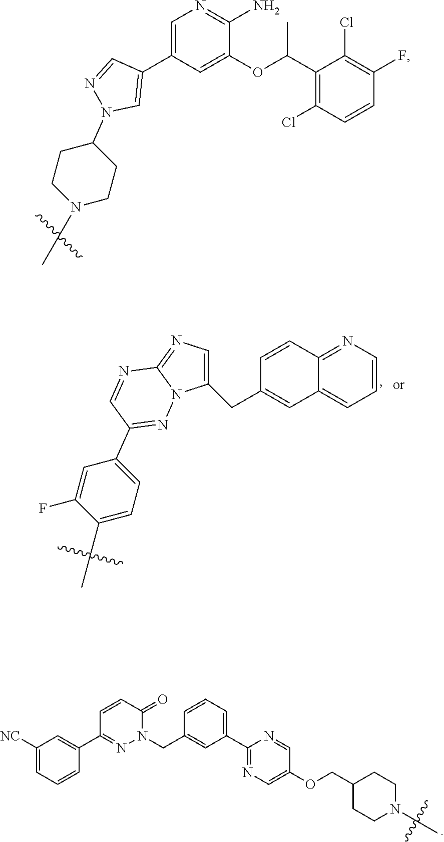 [SMILES: *N1CCC(n2cc(-c3cnc(N)c(OC(C)c4c(Cl)ccc(F)c4Cl)c3)cn2)CC1 ; *c1ccc(-c2cnc3ncc(Cc4ccc5ncccc5c4)n3n2)cc1F ; *N1CCC(COc2cnc(-c3cccc(Cn4nc(-c5cccc(C#N)c5)ccc4=O)c3)nc2)CC1]

4:
The compound of claim 1, wherein L is

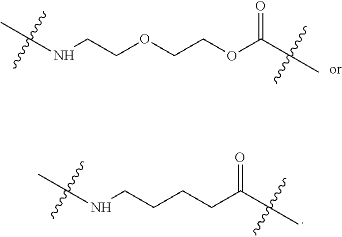 [SMILES: *NCCOCCOC(*)=O ; *NCCCCC(*)=O]

5: A compound of formula I selected from the group consisting of the following compounds:

| Com- | |
|---|---|
| pound | Structure |
| OZD- MET 01 | 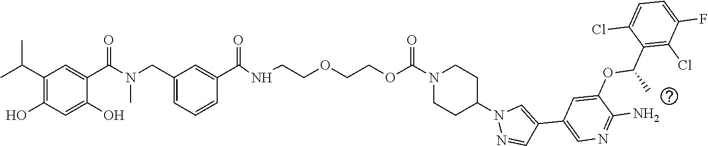 [SMILES: CC(C)c1cc(C(=O)N(C)Cc2cccc(C(=O)NCCOCCOC(=O)N3CCC(n4cc(-c5cnc(N)c(O[C@@H](C)c6c(Cl)ccc(F)c6Cl)c5)cn4)CC3)c2)c(O)cc1O] |
| OZD- MET 02 | 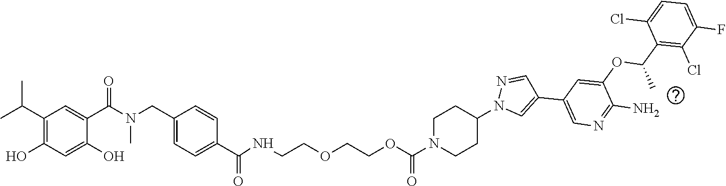 [SMILES: CC(C)c1cc(C(=O)N(C)Cc2ccc(C(=O)NCCOCCOC(=O)N3CCC(n4cc(-c5cnc(N)c(O[C@@H](C)c6c(Cl)ccc(F)c6Cl)c5)cn4)CC3)cc2)c(O)cc1O] |
| OZD- MET 03 | 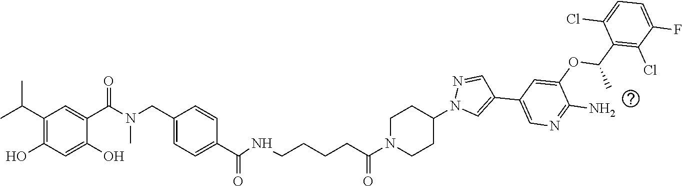 [SMILES: CC(C)c1cc(C(=O)N(C)Cc2ccc(C(=O)NCCCCC(=O)N3CCC(n4cc(-c5cnc(N)c(O[C@@H](C)c6c(Cl)ccc(F)c6Cl)c5)cn4)CC3)cc2)c(O)cc1O] |
| OZD- MET 04 | 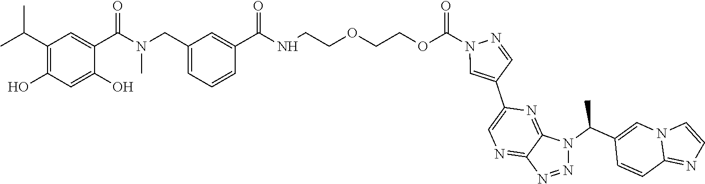 [SMILES: CC(C)c1cc(C(=O)N(C)Cc2cccc(C(=O)NCCOCCOC(=O)n3cc(-c4cnc5nnn([C@@H](C)c6ccc7nccn7c6)c5n4)cn3)c2)c(O)cc1O] |
| OZD- MET 05 | 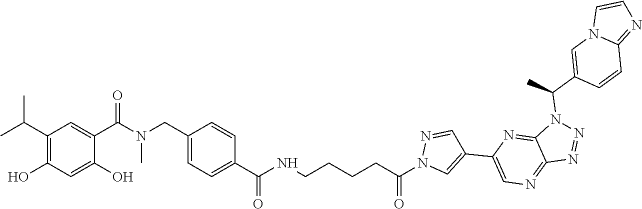 [SMILES: CC(C)c1cc(C(=O)N(C)Cc2ccc(C(=O)NCCCCC(=O)n3cc(-c4cnc5nnn([C@@H](C)c6ccc7nccn7c6)c5n4)cn3)cc2)c(O)cc1O] |
| OZD- MET 06 | 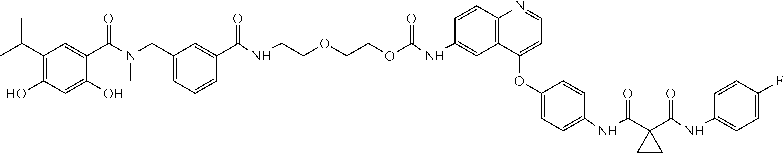 [SMILES: CC(C)c1cc(C(=O)N(C)Cc2cccc(C(=O)NCCOCCOC(=O)Nc3ccc4nccc(Oc5ccc(NC(=O)C6(C(=O)Nc7ccc(F)cc7)CC6)cc5)c4c3)c2)c(O)cc1O] |
| OZD- MET 07 | 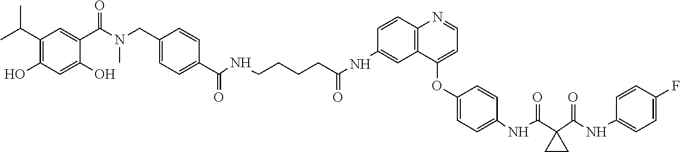 [SMILES: CC(C)c1cc(C(=O)N(C)Cc2ccc(C(=O)NCCCCC(=O)Nc3ccc4nccc(Oc5ccc(NC(=O)C6(C(=O)Nc7ccc(F)cc7)CC6)cc5)c4c3)cc2)c(O)cc1O] |
| OZD- MET 08 | 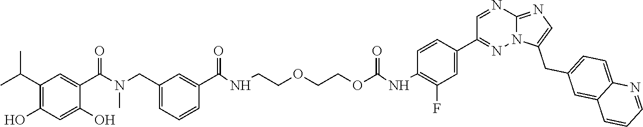 [SMILES: CC(C)c1cc(C(=O)N(C)Cc2cccc(C(=O)NCCOCCOC(=O)Nc3ccc(-c4cnc5ncc(Cc6ccc7ncccc7c6)n5n4)cc3F)c2)c(O)cc1O] |
| OZD- MET 09 | 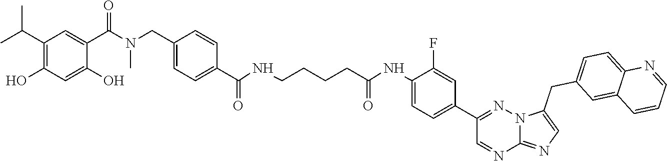 [SMILES: CC(C)c1cc(C(=O)N(C)Cc2ccc(C(=O)NCCCCC(=O)Nc3ccc(-c4cnc5ncc(Cc6ccc7ncccc7c6)n5n4)cc3F)cc2)c(O)cc1O] |
| OZD- MET 10 | 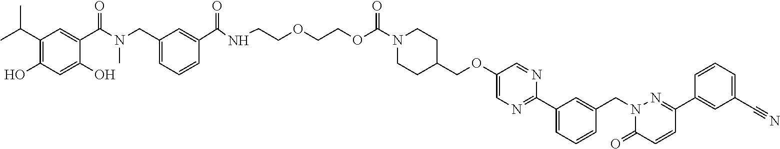 [SMILES: CC(C)c1cc(C(=O)N(C)Cc2cccc(C(=O)NCCOCCOC(=O)N3CCC(COc4cnc(-c5cccc(Cn6nc(-c7cccc(C#N)c7)ccc6=O)c5)nc4)CC3)c2)c(O)cc1O] |
| OZD- MET 11 | 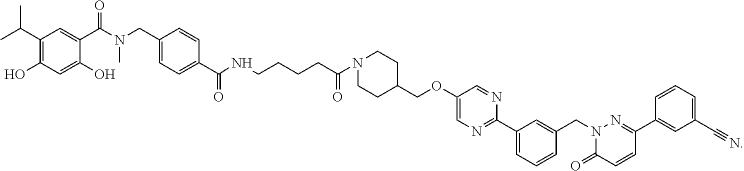 [SMILES: CC(C)c1cc(C(=O)N(C)Cc2ccc(C(=O)NCCCCC(=O)N3CCC(COc4cnc(-c5cccc(Cn6nc(-c7cccc(C#N)c7)ccc6=O)c5)nc4)CC3)cc2)c(O)cc1O] |

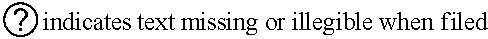

6: A pharmaceutical composition comprising the compound of formula 1 according to claim 1 and a pharmaceutically acceptable carrier.

7. (canceled)

8: A method for treating cancer in a subject in need thereof, comprising administering to the subject the compound of formula 1 according to claim 1 or a pharmaceutical composition comprising the compound of formula 1 according to claim 1 and a pharmaceutically acceptable carrier.

9: The composition of claim 6, wherein the cancer disease is selected from the group comprising acoustic neuroma, acute leukemia, acute lymphoblastic leukemia, acute myeloid leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchioloalveolar carcinoma, cervical cancer, chondrosarcoma, chordoma, choriocarcinoma, chronic leukemia, chronic lymphocytic leukemia, chronic myeloid (granulocytic) leukemia, chronic myelogenous leukemia, colorectal cancer, colon cancer, craniopharyngioma, cystadenocarcinoma, diffuse large B-cell lymphoma, Burkitt lymphoma, dysproliferative changes (dysplasia and metaplasias), embryonal carcinoma, endometrial cancer, endothelial sarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatic tumor, hepatocellular carcinoma, hormone-refractory prostate cancer, leiomyosarcoma, liposarcoma, lung cancer, lymphangioendothelioma, lymphangiosarcoma, lymphoblastic leukemia, lymphoma (Hodgkin and non-Hodgkin), malignancies and hyperproliferative disorders of the bladder, breast, colon, lung, ovary, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myeloid leukemia, myeloma, myxosarcoma, neuroblastoma, non-small cell lung cancer, oligodendroglioma, oral cancer, osteosarcoma, ovarian cancer, pancreatic cancer, papillary adenocarcinoma, papillary carcinoma, pinealoma, polycythemia vera, prostate cancer, rectal cancer, renal cell carcinoma, retinoblastoma, rhabdomyosarcoma, sarcomas, sebaceous carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumor, uterine cancer, Wilms' tumor, primary cancer, metastatic cancer, oropharynx cancer, hypopharyngeal cancer, liver cancer, gallbladder cancer, bile duct cancer, small intestine cancer, urinary tract cancer, kidney cancer, urothelial cancer, gynecologic cancers, uterine cancer, gestational trophoblastic disease, male genital cancer, seminal vesicle cancer, testicular cancer, germ cell tumor, endocrine tumor, thyroid cancer, adrenal cancer, pituitary cancer, hemangioma, sarcoma arising from bone and soft tissue, Kaposi's sarcoma, nerve cancer, eye cancer, meningeal cancer, glioblastoma, neuroma, neuroblastoma, schwannomas, solid tumors arising from hematopoietic malignancies such as leukemia, metastatic melanoma, recurrent or persistent ovarian epithelial cancer, fallopian tube cancer, primary peritoneal cancer, gastrointestinal stromal tumor, colon cancer, stomach cancer, melanoma, glioblastoma multiforme, non-squamous non-small cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharyngeal cancer, oral cancer, biliary tract, hepatocellular carcinoma, squamous cell carcinoma of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colon cancer, sarcoma, melanoma, renal carcinoma, endometrial cancer, thyroid cancer, rhabdomyosarcoma, multiple myeloma, ovarian cancer, glioblastoma, gastrointestinal stromal tumor, mantle cell lymphoma, and refractory malignant tumor.
